(19) United States
(12) Patent Application Publication
(10) Pub. No.: US 2024/0164822 A1
(43) Pub. Date: May 23, 2024

(54) CONTINUOUS FLOW BALLOON CATHETER SYSTEMS AND METHODS OF USE

(71) Applicant: PAVmed, Inc., New York, NY (US)

(72) Inventors: Lishan Aklog, New York, NY (US); Brian J. deGuzman, New York, NY (US); Richard Yazbeck, New York, NY (US); Michael Boutillette, New York, NY (US); Jim Pelletier, New York, NY (US); Liem Vu, New York, NY (US); Mack Krell, New York, NY (US); Minh Doung, New York, NY (US); Gregory Eberl, New York, NY (US)

(21) Appl. No.: 18/282,836

(22) PCT Filed: Mar. 21, 2022
(86) PCT No.: PCT/US22/21114

§ 371 Date: Sep. 19, 2023

Related U.S. Application Data
(60) Provisional application No. 63/164,226, filed on Mar. 22, 2021.
(60) Provisional application No. 63/175,712, filed on Apr. 16, 2021.

(51) Int. Cl. A61B 18/04; A61B 18/00; A61M 25/10

(52) U.S. Cl. CPC A61B 18/04; A61M 25/10184; A61B 2018/0022; A61B 2018/00577; A61B 2018/046

(57) ABSTRACT

Systems and methods for treatment of tissue including an expandable member designed to be positioned at a site of interest against tissue to be treated and to accommodate fluid set at a temperature range to affect the tissue at the site of interest; an assembly for directing the fluid set at the temperature range into the expandable member, the assembly having an input to introduce the fluid into the expandable member to circulate substantially along an entire length of the expandable member and an output spatially situated from the input to permit fluid flow to be removed from within the expandable member; and a mechanism designed to direct fluid along the assembly and through the input into the expandable member while simultaneously removing fluid from the expandable member through the output, so as to maintain substantially constant fluid pressure within the expandable member.

### CROSS-REFERENCE TO RELATED APPLICATIONS

This application claims priority to, and the benefit of, U.S. Provisional Application No. 63/164,226, filed Mar. 22, 2021, and U.S. Provisional Application No. 63/175,712, filed Apr. 16, 2021, for all subject matter common to both applications. The disclosure of said provisional applications are hereby incorporated by reference in its entirety.

### FIELD

The disclosure relates generally to systems and methods for infusion or ablation target tissues with a balloon catheter.

### BACKGROUND

Balloon catheters are used for a wide variety of medical applications including angioplasty, stent deployment, embolectomy, and balloon occlusion of blood vessels. A standard balloon catheter has a catheter with at least one lumen, a compliant or non-compliant balloon positioned coaxially around and bonded to the catheter at or near its distal tip. At least one of the catheter lumens, the inflation lumen, has at least one orifice positioned within the balloon lumen such that this inflation lumen is in fluid communication with the inside of the balloon. The balloon is deployed by attaching a syringe or other infusion device to the proximal end of the catheter, so that it is in fluid communication with the catheter's inflation lumen and injecting a volume of fluid (liquid or gas) through the inflation lumen into the balloon, inflating it to a given volume or pressure. The balloon is deflated by withdrawing the fluid from the balloon lumen through the catheter's inflation lumen back into the reservoir of the syringe or other infusion device. The catheter may have additional lumens such as a guidewire lumen to facilitate maneuvering of the catheter within the body, infusion lumens to infuse fluid out the distal tip of the catheter into the patient and monitoring lumens to monitor pressure, temperature, or other parameters.

There are applications where it is desirable for the fluid which inflates the balloon to flow continuously into and out of the balloon while maintaining the balloon inflated at the desired volume and pressure. One such application would be thermal ablation balloon catheters which ablate tissue using hyper or hypothermia. Balloon catheters are useful in these applications because they can be designed to conform to the tissue to be ablated once positioned in the appropriate location. Another such application would be a drug delivery balloon catheter where the balloon serves as a reservoir for a drug to be delivered through its permeable wall.

Tissue ablation is performed throughout the body. It is frequently used to destroy abnormal tissue such as malignant tumors (e.g., liver, lung) or other non-malignant tissue (e.g., endometrial, prostatic). It is also frequently used to target structurally normal tissues for a specific therapeutic effect such as cardiac tissue ablation to treat arrhythmias and more recently renal nerve ablation (“renal denervation”) to treat refractory hypertension.

Tissue ablation is most commonly performed by applying energy to the target tissue to cause irreversible cellular injury. Common energy sources for tissue ablation include radiofrequency, microwave, laser, ultrasound and cryo. Each source has its own specific characteristics, biophysical mechanism, advantages, and disadvantages. All of these modalities, with the exception of cryo, ultimately act by increasing the tissue temperature to cytotoxic levels for a given period of time. Cellular injury is generally reversible below 46° C. Although there is some variability in thermal sensitivity among different tissues and cell types, irreversible cellular injury generally occurs after 60 minutes at 46° C. and less than 5 minutes at 50° C.

Most clinical applications of thermal ablation have involved either large volumes of tissue (e.g., tumor ablation) or at least relatively thick tissues (e.g., cardiac ablation) where complete ablation of the target tissue is necessary for a successful therapeutic effect. Even a small volume of residual viable tissue can lead to clinical failure in the form of recurrent tumor growth, metastases from residual tumor or recurrent arrhythmias from residual pathways. For the ablation to be successful, the cells farthest from the energy source must reach the target cytotoxic temperature. The larger the distance from the energy probe to the border of the target tissue the more challenging the ablation, the more energy needs to be delivered and the higher the temperature near the probe needs to be. For example, RF ablation depends on electrical conductivity to generate heat but creating too much heat near the probe can generate charring which increases impedance and decreases the effective range of the ablation. A wide variety of technologies and techniques have been developed to accommodate the challenges of ablating across a large distances using RF (e.g., multi-electrode probes, cooling, irrigation, and complex power algorithms). As a result, these tissue ablation modalities typically require a complex, external console to assure the precise amount of energy is delivered to the tissue to achieve the desired therapeutic effect. Simpler devices which use a “shotgun” approach may be ineffective or downright harmful.

The major limitation of standard balloon catheters in hyperthermic ablation applications is that the surrounding tissue serves as a powerful thermal sink. The temperature in the balloon may equilibrate with the surrounding tissue within a short period of time, shorter than the time necessary to perform the ablation, typically several minutes. For hypothermic (cryo) ablation the fluid temperature can be made so cold using liquid gases (e.g., argon, nitrogen) that the time required for the temperature to equilibrate is longer than the time it takes to ablate the tissue. For hyperthermic ablation, however, the options are more limited since the boiling temperature of most biocompatible fluids are only modestly above the temperature necessary to successfully ablate most tissues. Most tissue ablation is therefore performed using a fixed probe which is inserted into the tissue and attached to an external energy source (e.g., radiofrequency, microwave). The source continuously provides energy to the tissue as the heat dissipates into the surrounding tissue.

### SUMMARY

In accordance with example embodiments of the present disclosure, a system for treatment of tissue is provided herein. The system include an expandable member designed to be positioned at a site of interest against tissue to be treated and to accommodate fluid set at a temperature range to affect the tissue at the site of interest; an assembly for directing the fluid set at the temperature range into the expandable member, the assembly having an input to introduce the fluid into the expandable member to circulate substantially along an entire length of the expandable member and an output spatially situated from the input to permit fluid flow to be removed from within the expandable member; and a mechanism designed to direct fluid along the assembly and through the input into the expandable member while simultaneously removing fluid from the expandable member through the output, so as to maintain substantially constant fluid pressure within the expandable member.

In accordance with aspects of the present disclosure, the assembly can include an input fluid pathway and an output fluid pathway, where the input fluid pathway can extend from the mechanism to the input and the output fluid pathway can extend from the mechanism to the output. The input fluid pathway can be disposed concentrically with the output fluid pathway. The output fluid pathway can be arranged between an inner surface of an elongated member and an outer surface of the input fluid pathway. The input fluid pathway can extend into the expandable member to deliver fluid into the expandable member, and a proximal end of the expandable member is partially disposed within the output fluid pathway.

In accordance with aspects of the present disclosure, the proximal end of the expandable member can be adhered to an inner surface of the elongated member. A portion of the input fluid pathway that can be disposed within the expandable member defines a plurality of inflow ports adjacent a distal end of the input fluid pathway. The plurality of inflow ports can be arranged in a plurality of sets, each of the plurality of sets being axially spaced from others of the plurality of sets. Each of the plurality of sets can be circumferentially misaligned with adjacent ones of the plurality of sets.

In accordance with aspects of the present disclosure, the system can further include an elongated member extending from a proximal end in fluid communication with the input and output to a distal end in fluid communication with the expandable member, where the input and output can extend through the elongated member. The system can further include a plug disposed on a distal end of the expandable member. The expandable member can be a treatment balloon which can be configured to permit for heat transfer between the fluid and a target to ablate at least a portion of the target. The expandable member can be a treatment balloon, where the system can further include an outer balloon disposed around the treatment balloon and the outer balloon can be in fluid communication with a fluid pathway to expand the outer balloon with a fluid. The outer balloon can be configured to expand against surrounding tissue to thereby anchor the treatment balloon in place within the site of interest. The treatment balloon can be configured to rotate or translate relative to, and independent of, the outer balloon. The system can further include an endoscope having a lumen extending therethrough. The expandable member can be configured to rotated relative to, and independent of, the endoscope.

In accordance with example embodiments of the present disclosure, a method of treating tissue is provided herein. The method includes positioning, adjacent a target tissue to be treated, an expandable member having an assembly in fluid communication therewith for directing fluid into the expandable member, the assembly having an input to introduce the fluid into the expandable member and an output spatially situated from the input to permit fluid flow to be removed from within the expandable member; infusing fluid into the expandable member, through the input of the assembly, so as to enlarge the expandable member to retain the expandable member in at least one longitudinal position against the target tissue; and continuing to direct, through the input of the assembly, fluid into the expandable member and along its length to allow the fluid to transfer its energy to the tissue while simultaneously removing, through the output of the assembly, fluid from the expandable member, so as to maintain substantially constant fluid pressure within the expandable member.

In accordance with aspects of the present disclosure, the assembly can include an elongate member and the method can further include, advancing the elongate member through a lumen of an endoscope, and rotating at least the expandable member independent of the endoscope to treat the target tissue while maintaining a stationary view through the endoscope. The method can further include expanding an outer expandable member, disposed about the expandable member to contact surrounding tissue at a pressure sufficient to anchor the elongate member in place within a lumen in a patient proximate to the tissue. The method can further include rotating the expandable member independent of the outer expandable member to treat additional tissue about a circumference of the lumen. The method can further include translating the expandable member relative to the outer expandable member to treat additional tissue along a length of a tract. Thermal energy from the fluid directed into and out of an interior of the expandable member can treat the target tissue through the expandable member and the outer expandable member.

In accordance with aspects of the present disclosure, the expandable member can further include a distal balloon, where the method can further include either: expanding the distal balloon to contact surrounding tissue at a pressure sufficient to anchor the expandable member in place within the body, or expanding the distal balloon to dimensions greater than a diameter of a passageway within a body such that, when positioned beyond the passageway and expanded, such that the distal balloon is prevented from being pulled back through the passageway. Treating the target tissue can include treating tissue in an upper gastrointestinal tract; treating tissue in a lower gastrointestinal tract; treating parasympathetic nerves in a renal artery; treating parasympathetic nerves in a nasal cavity; treating vessels in a circulatory system; treating fistulae tracts; and/or treating breast tissue.

In accordance with example embodiments of the present disclosure, a system for treatment of tissue is provided herein. The system includes an expandable member sized to be positioned against tissue to be treated and to accommodate fluid set at a range of temperatures to affect the tissue; an assembly extending into the expandable member for directing the fluid thereinto, the assembly including, a first fluid path terminating at an input so as to permit fluid introduce into the expandable member to circulate along its length; a second fluid path terminating in an output, and is spatially situated from the input to permit circulated fluid flow to be removed from within the expandable member; and a mechanism, in fluid communication with the assembly, designed to push fluid through the input into the expandable member while simultaneously remove fluid from the expandable member through the output, so as to maintain substantially constant fluid pressure within the expandable member.

In accordance with aspects of the present disclosure, the expandable member can be disposed an inner surface of the second fluid path. The second fluid path can be disposed around the first fluid path and is concentric with the first fluid path. A portion of an outer surface of the expandable member can be adhered to an inner surface of the second fluid path.

In some aspects, the system can further include a plug disposed adjacent a distal end of the expandable member. The plug can be coupled to at least one of the expandable member and the assembly. The plug can be coupled to the expandable member and translatable relative to the assembly. The plug can be cup-shaped and includes a base and a circumferential sidewall, the plug being configured to receive at least a portion of the assembly. The plug can be unitarily formed with the expandable member.

The first fluid path can define a plurality of inputs adjacent a distal end thereof, the plurality of inputs being arranged in a plurality of sets, each of the plurality of sets being axially spaced from others of the plurality of sets. The plurality of sets can include two sets of inputs. Each of the two sets of inputs can include multiple inputs. Each of the two sets of inputs can include two inputs disposed 180 degrees apart. Each of the two sets of inputs can include three inputs disposed 120 degrees apart.

### BRIEF DESCRIPTION OF THE DRAWINGS

FIG. 1 illustrates a continuous flow balloon catheter system in accordance with an embodiment of the present invention.

FIG. 2A illustrates a balloon catheter in accordance with an embodiment of the present disclosure.

FIG. 2B is a crop sectional view of a balloon catheter illustrating the various layers and lumens within the balloon catheter.

FIG. 2C and FIG. 2D are crop sectional views of a balloon catheter illustrating thermal insulation of flow lumens.

FIG. 3 illustrates a representative embodiment of a balloon catheter system for treating tissue within the body in accordance with the present disclosure.

FIG. 4 illustrates a representative embodiment of balloon catheter system being used to treat target tissue in accordance with the present disclosure.

FIG. 5 depicts a side cross-sectional view of an elongate member and balloon in accordance with various embodiments of the present disclosure.

FIG. 6 illustrates a front cross-sectional view of an elongate member and balloon in accordance with various embodiments of the present disclosure.

FIG. 7 illustrates a front cross-sectional view of an elongate member and balloon, showing a balloon in deflated and inflated states.

FIG. 8 illustrates an embodiment of a balloon catheter system with a balloon positioned on a distal end of an elongate member beyond the passageway in accordance with the present disclosure.

FIG. 9 is a side cross-sectional view of a distal portion of a balloon catheter system to show how a balloon is inflated and deflated.

FIG. 10 is a front cross-sectional view of a distal portion of a balloon catheter system to show how a balloon is inflated and deflated.

FIG. 11 is a front cross-sectional view of a distal portion of a balloon catheter system showing a balloon in deflated and inflated states.

FIG. 12 illustrates a representative embodiment of another balloon catheter system for treating tissue within the body in accordance with the present disclosure.

FIG. 13 is a side cross-sectional view of a distal portion of a balloon catheter system in accordance with various embodiments of the present disclosure.

FIG. 14 is a side cross-sectional view of a distal portion of a balloon catheter system in accordance with various embodiments of the present disclosure.

FIG. 15 is a front cross-sectional view of a distal portion of a balloon catheter system showing an inner balloon and outer balloon in deflated and inflated states.

FIG. 16 illustrates a representative embodiment of a balloon catheter system with a balloon positioned on a distal end of an elongate member beyond the passageway in accordance with the present disclosure.

FIG. 17 is a side cross-sectional view of a distal portion of a balloon catheter system to show how a balloon is inflated and deflated.

FIG. 18 is a front cross-sectional view of a distal portion of a balloon catheter system to show how a balloon is inflated and deflated.

FIG. 19 is a front cross-sectional view of a distal portion of a balloon catheter system showing an outer balloon in deflated and inflated states, and an inner balloon and anchor balloon in inflated states.

FIGS. 20A, 20B, 21A, 21B, 22A, 22B, 23A, and 23B depict various embodiments of a balloon catheter system in which an inner balloon is configured to rotate and/or translate independent of an outer balloon.

FIGS. 24A-24B and 25A-25B illustrate various embodiments of another balloon catheter system having two or more balloons spaced apart a circumference of an elongate member.

FIGS. 26A-26B illustrate a representative embodiment of a balloon catheter system in which one or more conduits are arranged in a concentric manner.

FIGS. 27A-27C, 28A-28B, and 29A-29C illustrate representative techniques for streamlining a profile of balloon systems of the present disclosure to facilitate advancement to the treatment site in a tract.

FIGS. 30A-30B, 31A-31B, and 32A-32B illustrate various additional embodiments of balloon catheter systems of the present disclosure.

FIGS. 33A-33B illustrate various additional embodiments of the balloon catheter systems of the present disclosure.

FIG. 34 is a side cross-sectional view of an alternative balloon catheter system of the present disclosure.

FIG. 35 is a side cross-sectional view of another alternative balloon catheter system of the present disclosure.

FIGS. 36A and 36B are front cross sectional views of two alternative balloon catheter systems of the present disclosure.

FIGS. 37A-37C illustrate various configurations of an infusion device in accordance with an embodiment of the present disclosure.

### DETAILED DESCRIPTION

In general, there are many applications where it is desirable for a fluid, which can inflate a balloon or expandable member, to continuously flow from an assembly into and out of the balloon while maintaining the balloon inflated at a desired volume and pressure to assure continuous tissue contact. At the same time, it is often desirable to ensure proper flow of the fluid within the balloon, with the assembly. For example, the assembly can be configured to ensure that the fluid flows, or circulates, substantially along an entire length of the balloon from an input to an outlet. As will be outlined below, the balloon, or expandable member, can have a variety of configurations which may be of particular use in a given procedure. The assembly for directing the fluid can provide fluid to the balloon at a predetermined temperature range needed for a medical procedure. For example, the fluid can be maintained at the predetermined temperature range and can be used for thermal treatment of tissue in contact with the balloon. In an effort to ensure that the fluid is directed through substantially a majority of the length of the balloon for a given treatment, the assembly can, in general, include at least one input and at least one output, which are spatially situated from one another to permit the fluid to flow, or circulate, along the entire length of the balloon to allow for the desired thermal treatment. The assembly for directing fluid can include at least a mechanism in fluid communication with the assembly that can be designed to push fluid through the input into the expandable member while simultaneously removing fluid from the expandable member through the output, so as to maintain substantially constant fluid pressure within the expandable member. This general concept is applicable in all of the foregoing embodiments, described herein.

One such application of this general concept can be in the field of thermal ablation balloon catheters which ablate tissue using hyperthermia or hypothermia. In such applications the surrounding tissues serve as a heat sink which rapidly dissipates thermal energy from the balloon or radiates thermal energy into the balloon. A possible solution to the limitation of balloon catheters equilibrating with their surrounding tissues is to circulate a hot or cold fluid into and out of a balloon while maintaining the balloon at an inflation which is critical to assure tissue contact and thermal transfer into a target tissue. Maintaining such an equilibrium requires continuous flow with precise matching of flow into and out of the balloon. Further, it is imperative to ensure that the circulation of fluid flow is consistent along substantially the entire length of the balloon, in the proximal to distal direction. Thus, the fluid input and output can be strategically spaced to ensure that the flow of fluid circulates along an entire length of the expandable member to provide for a consistent thermal profile along the entire expandable member.

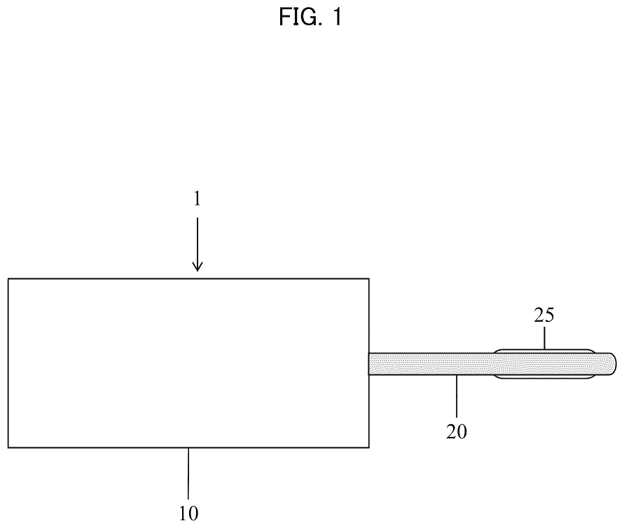

Therefore, in accordance with an embodiment of the present disclosure, a balloon catheter system 1 is provided with an expandable member 25, e.g., a treatment balloon, having continuous flow of a fluid into and out through an assembly 20, e.g., a catheter, and an infusion mechanism or device 10, for pushing (i.e., directing) fluid into expandable member 25 and withdrawing fluid from expandable member 25, as illustrated in FIG. 1. It should be appreciated that FIG. 1 is intended to provide a conceptual overview of the instant disclosure and is not representative of a specific embodiment, as will be detailed below. In general, the infusion device 10 may continuously drive or recirculate a fluid into and out of an expandable member through the assembly 20 while maintaining the expandable member 25 inflated at a specified volume and pressure. In some embodiments, the fluid may be replenished or replaced for each given cycle. Alternatively, in some embodiments the fluid can be recirculated or recycled. In some embodiments, the infusion device 10 may first heat the fluid (or liquid) to a target temperature, then continuously drive or recirculate the heated fluid, through the assembly 20, into and out of the expandable member 25, while also maintaining the expandable member 25 inflated to a specified volume and pressure.

Turning to the shared details of the expandable member 25, or the various balloons, expandable member 25 may be constructed of any compliant, semi-compliant or non-compliant material, typically a plastic such as polyurethane, nylon, polyethylene, PET or PEBAX, or any material that permits member 25 to be enlarged. In some embodiments, the assembly 20 may carry heated liquid into the expandable member 25 via an inlet, discussed below with respect to specific embodiments. The heated fluid can thereafter be circulated substantially along the length of the expandable member 25, and the assembly 20 can then carry the liquid out of the expandable member 25 via an outlet, again discussed below with respect to specific embodiments. Additionally, the fluid can be continuously, and simultaneously, circulated into and out of the expandable member 25 at the same time.

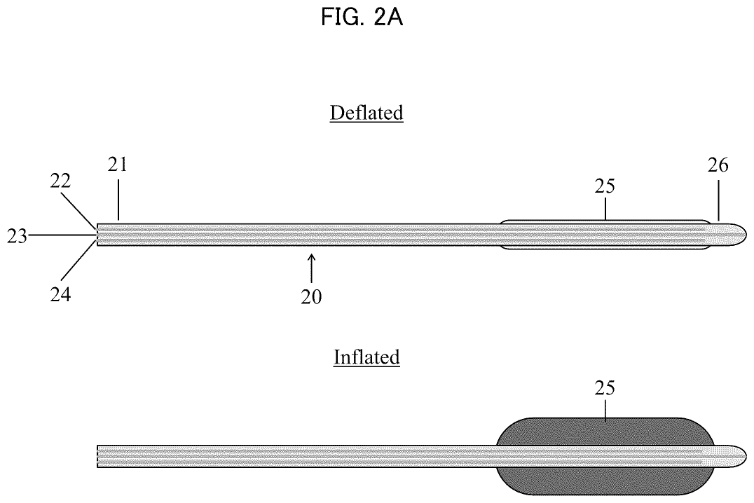

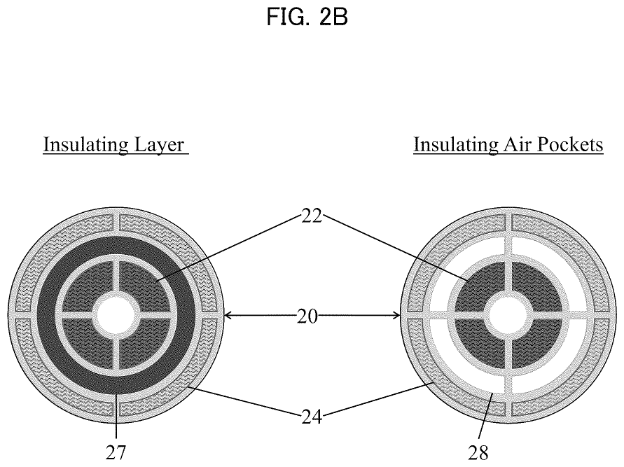

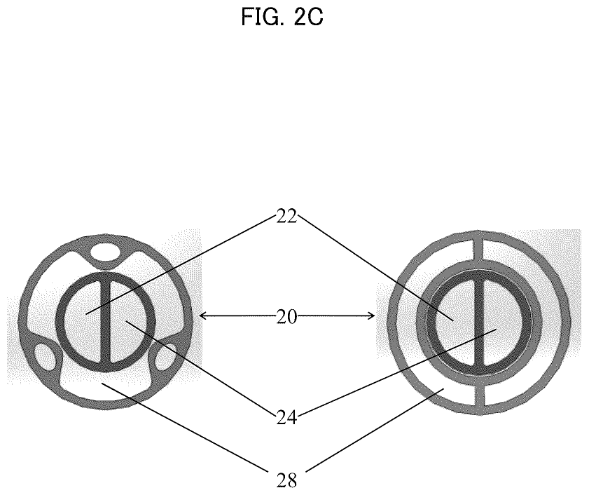

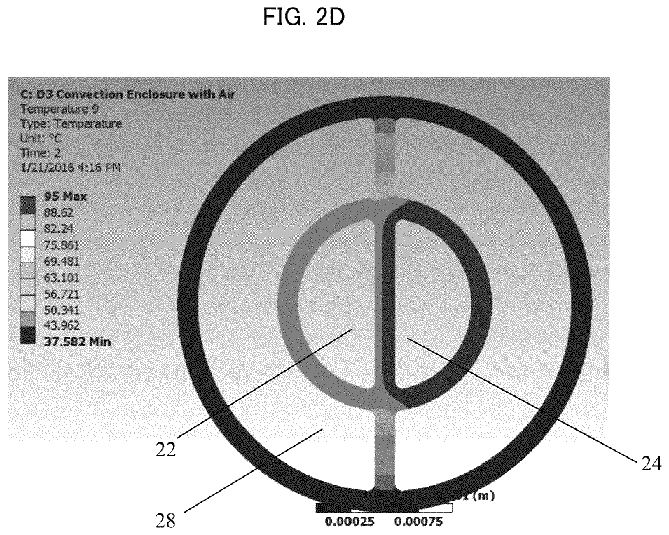

The assembly 20, or fluid delivery assembly, can, in some embodiments, provide any number of lumens 22, 24 spatially situated within the assembly 20 to minimize thermal transfer between the inflowing and outflowing fluid streams, as well as between these streams and a patient's blood and tissues, shown conceptually in FIG. 2A. Alternatively, in some embodiments, the lumens, or flow paths, can be concentric with one another such that one flow path surrounds another flow path. In further alternatives, or in addition, more than two lumens, or flow paths, may be included in the assembly—depending on the specific design and implementation of the expandable member. The catheter 20 may also have additional features to minimize thermal loss such as a thermal insulating material 27 or air pockets 28 (as seen in FIGS. 2B, 2C, and 2D) surrounding the inflow lumens 22-24, such that the flow lumens 22, 24 are thermally insulated and may have different temperatures (as seen in FIG. 2D, where the inflow lumen 22 carrying heated fluid can be at a different temperature than the outflow lumen 24).

The assembly 20 may be a catheter, in some embodiments, that can be made of similar materials to the expandable member 25. In various embodiments, the assembly 20 can comprise at least two or more flow lumens 22-24, each in fluid communication with the expandable member 25 through one or more distal orifices. When the system 1 is active, one or more inflow lumens 22 carries fluid into the balloon 25 and one or more outflow lumens 24 carries fluid out of the balloon 25. The system 1 can be designed so that flow of the fluid can be reversed with each flow lumen 22, 24 serving as either inflow 22 or outflow 24 depending on the direction of flow. In some embodiments, when the flow is reversed, the inflow lumen 22 will become the outflow lumen, and the outflow lumen 24 will become the inflow lumen. In some embodiments, the catheter 20 may contain additional lumens as desired for guidewires, infusion, monitoring, and other functionalities that may be directed via the additional lumens.

As noted above, the general concept of driving, or circulating, a fluid along substantially the entire length of the expandable member 25 can be implemented with a number of different expandable members and a number of different fluid delivery assemblies. The following disclosure provides for a number of implementations, where one of ordinary skill in the art would readily understand to be combinable in any combination. For example, an expandable member of one embodiment may be used with the fluid delivery assembly of a different embodiment. With that in mind, the following descriptions of expandable members in combination with fluid delivery assemblies, or balloon catheter systems, are provided below.

### Balloon Catheter System 3100

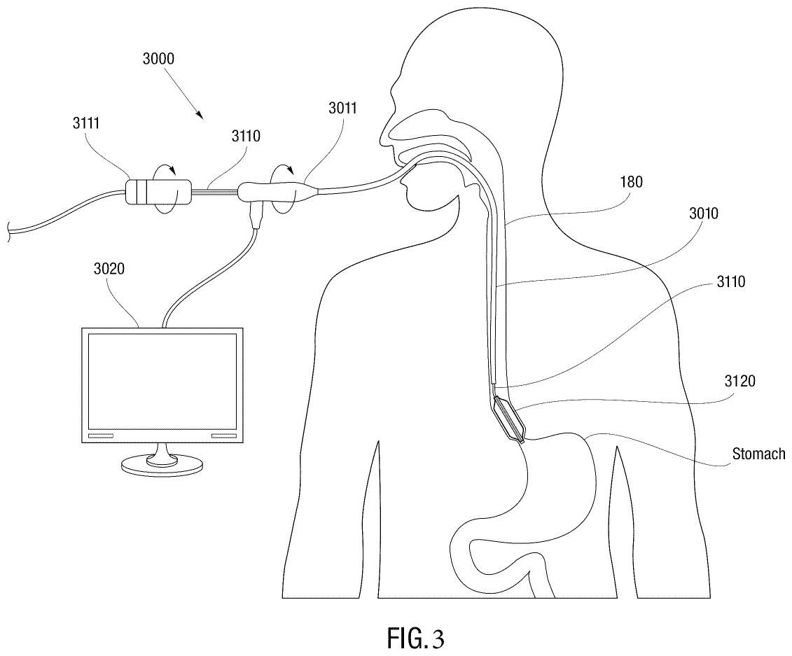

Turning to FIGS. 3-7, a representative embodiment of a balloon catheter system 3100 for treating tissue within the body is illustrated. System 3100, in various embodiments, may generally include an assembly 3110 (“elongate member”) and an expandable member 3120 (“balloon”) positioned near a distal end of elongate member 3110. System 3100, as illustrated in FIGS. 3-7, can be used in connection with an endoscope 3010 and a display system, such as monitor 3020 to visualize a site of interest. Generally speaking, endoscope 3010 may be positioned within a tract 180 of the body (shown here as the digestive tract) at the site of interest and balloon 3120 advanced therethrough to treat the target tissue 182. Using monitor 3020, an operator can visualize the placement of balloon 3120 within body tract 180 and treatment of tissue therein. As will be appreciated by one of ordinary skill in the art, any suitable combination of endoscope and monitor may be used with the instant balloon catheter system 3100, or any of the following embodiments. While endoscope 3010 and monitor 3020 are illustrated herein, they are merely representative components and any endoscope, or monitor, known in the art may be used with the instant disclosure.

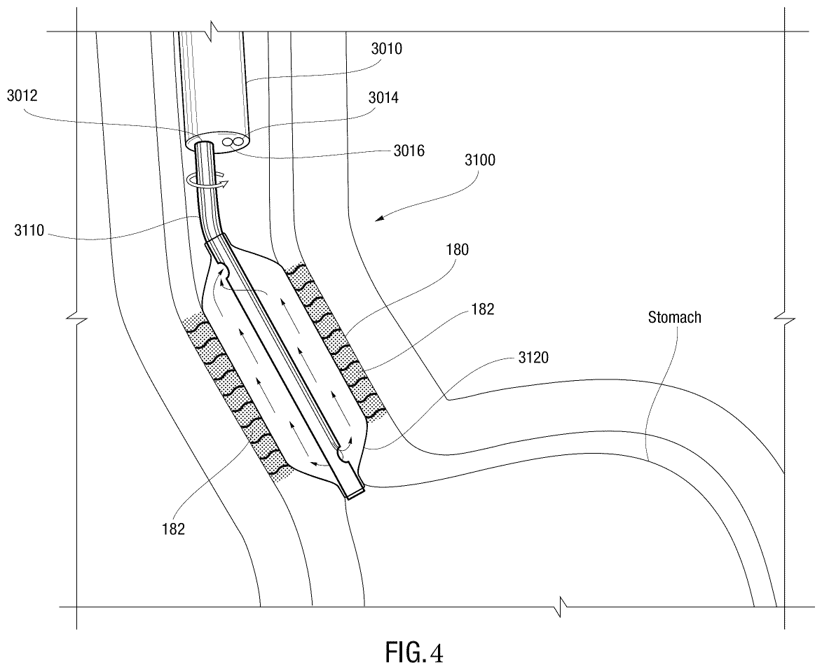

Balloon 3120, illustrated in FIGS. 4, may comprise any expandable member suitable for containing fluid directed into its interior through the conduits of elongate member 3110. In an embodiment, balloon 3120 may be configured to expand when fluid is introduced into its interior and to deflate when fluid is withdrawn therefrom. Balloon 3120, in various embodiments, may have a thickness sufficient to maintain structural integrity under associated pressures and temperatures, but also be thin enough or have other properties suitable to allow for thermal energy to transfer between hot or cold fluid in its interior and the tissue with which balloon 3120 is in contact. One of ordinary skill in the art will recognize suitable balloon sizes, shapes, and constructions for the purposes described herein.

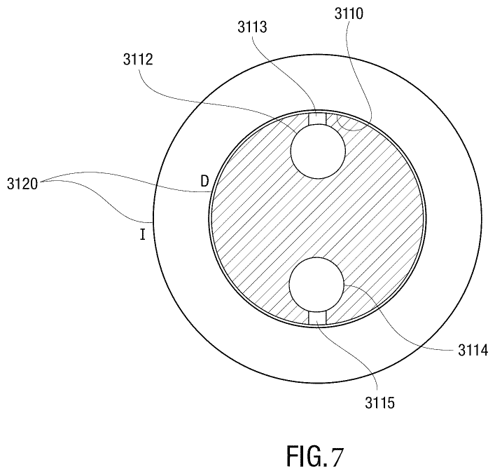

FIG. 7 depicts a front cross-sectional view of elongate member 3110 and balloon 3120 which show balloon 3120 in deflated (labeled “D”) and inflated (labeled “I”) states. Balloon 3120, in various embodiments, may be inserted into the body in a deflated state to make it easier to safely navigate balloon 3120 into position adjacent a target tissue 182 to be treated. In particular, in various embodiments, elongate member 3110 and balloon 3120 may be dimensioned such that these components, when balloon 3120 is deflated and collapsed against elongate member 3110 can be advanced through lumen 3012 of endoscope 3010 and to emerge therefrom proximate target tissue 182. Once positioned adjacent target tissue 182, deflated balloon 3120 may be inflated or enlarged by directing the fluid into and out of balloon 3120 through inflow and outflow conduits 3112, 3114, respectively. This may serve to both inflate balloon 3120 and treat adjacent tissue with thermal energy, as previously described. Upon completion of the treatment, balloon 3120 may be deflated by ceasing directing fluid into balloon 3120 and by withdrawing any remaining fluid from balloon 3120 if necessary. Deflated balloon 3120 may then be withdrawn back through lumen 3012 of endoscope 3010 for removal from the body.

Elongate member 3110, in various embodiments, may include any elongate structure suitable for directing balloon 3120 to a treatment site in tract 180, such as a catheter. To that end, in various embodiments, elongate member 3110 may be sufficiently flexible radially to navigate through bends and curves in digestive tract 180 while also being sufficiently stiff or rigid axially to be advanced through tract 180 and/or endoscope 3010 when pushed from its proximal end. Elongate member 3110, in various embodiments, may include or support one or more conduits 3112, 3114 running along its length through which fluid(s) may be directed into and out of balloon 3120 and any additional balloons. As shown throughout the present figures, these conduits may be internal to elongate member 3110. For example, in an embodiment, these conduits may be in the form of lumens extending within elongate member 3110, while in another embodiment, these conduits may be tubes positioned within a hollow-bodied elongate member 3110. In other embodiments, rather than extending within elongate member 3110, these conduits may instead extend along an outer surface of elongate member 3110 (not shown). For example, in an embodiment, the conduits may be tubes coupled to or otherwise disposed on the outer surface of elongate member 3110. It should be recognized that it may be preferable to direct hot or cold fluids through conduits running within elongate member 3110 as internal conduits may be more insulated against thermal transfer with surrounding tissue and bodily fluids than conduits running along an outer surface of elongate member 3110.

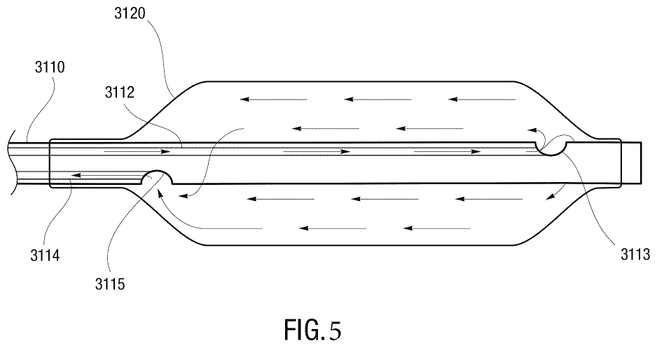

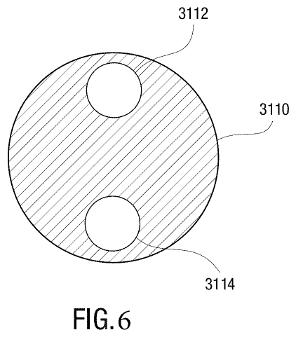

FIGS. 5-7 illustrate end cross-sectional and side views of elongate member 3110 and balloon 3120 to show how the fluid can be directed in and out of balloon 3120. In various embodiments, fluid may be directed into balloon 3120 through an inflow conduit 3112 from a fluid source and out of balloon 3120 through an outflow conduit 3114. Inflow conduit 3112 and outflow conduit 3114 may run within (as shown in FIG. 6) or along (as shown in FIG. 5) elongate member 3110 and may be placed into fluid communication with an interior portion of balloon 3120 via inflow port 3113 and outflow port 3115, respectively. In the embodiment shown in FIG. 5, inflow port 3113, can be situated near a distal end of balloon 3120 and outflow port 3115 can be spatially situated, from the inflow port 3113, near a proximal end of balloon 3120 such that fluid can be directed into and out of balloon 3120 from the distal end towards the proximal end. Advantageously, the flow of the fluid can be able to circulate, or be driven, along substantially the entire length of the balloon 3120 from near the proximal end to near the distal end. This circulation of fluid permits for substantially even heat distribution of the fluid flowing through the balloon 3120 to effectively treat any tissue in contact with the balloon 3120. Of course, the locations of inflow port 3113 and outflow port 3115 may be reversed such that fluid is directed into and out of balloon 3120 from the proximal end towards the distal end to similar effect. It should be recognized that by spacing out inflow port 3113 and outflow port 3115 to be located near opposing ends of balloon 3120, the fluid is encouraged to travel along the length of balloon 3120 and out via outflow port 3115, and thereby maintain a substantially uniform temperature distribution along the length of balloon 3120. This may make it easier to control the treatment since the operator would know that all tissue adjacent to balloon 3120 is receiving substantially the same dose of thermal energy. In other words, the rate of heat transfer along the entire length of the balloon 3120 can remain constant due to the circulation of fluid therethrough.

With reference again to FIGS. 3 and 4, endoscope 3010 may include a lumen 3012 extending therethrough, as well as a camera 3014 and a light 3016 near the distal end for visualizing nearby portions of the body tract 180. A handle 3011 may also be provided for facilitating placement and operation of endoscope 3010 within body tract 180, as shown in FIG. 3. For example, an operator may grip handle 3011 and either push or pull it to position endoscope 3010 within body tract 180. Likewise, an operator may grip handle 3011 and twist it to rotate endoscope 3010 within body tract 180. In some embodiments, such movement of endoscope 3010 may be decoupled from that of elongate member 3110 and balloon 3120, thereby allowing the operator to position camera 3014 and light 3016 in a location and orientation that best allows the operator to see body tract 180, target tissue 182, balloon 3120, or other features of interest as the case may be during a given step of the procedure. Likewise, as shown in FIG. 4, such decoupling may allow for endoscope 3010 to be held in a fixed position and orientation while elongate member 3110 and balloon 3120 are independently positioned, rotated, and otherwise manipulated, using handle 3111 of the balloon catheter system 3100 (which can be independent of the endoscope handle 3011) to treat tissue beyond endoscope 3010. It should be appreciated that although endoscope 3010 is described in connection with FIGS. 3 and 4, other endoscopes and associated features can be used in connection with the balloon catheter system of the present invention.

With reference now to FIG. 4, there is illustrated a representative embodiment of balloon catheter system 3100 being used to treat target tissue 182, here comprising tissue at the lower end of the esophagus near its juncture with the stomach. It should be recognized that system 3100 may additionally or alternatively be configured to treat a variety of other portions of the body and ailments such as, without limitation, treating tissue in the upper gastrointestinal tract, treating tissue in the lower gastrointestinal tract, ablating parasympathetic nerves in the renal artery, ablating parasympathetic nerves in the nasal cavity, ablating vessels in the circulatory systems, ablating fistulae tracts, and treating breast tissue.

As illustrated, endoscope 3010 may be positioned upstream of the target tissue 182 in a location and orientation that allows the operator to the treatment site and thus help guide balloon 3120 into position adjacent target tissue 182. Once positioned, hot or cold fluid may be continuously advanced through balloon 3120, causing thermal energy from the fluid to radiate through balloon 3120 and into the adjacent target tissue 182, as illustrated in FIG. 4. In some embodiments, a heating mechanism may heat the fluid to a temperature sufficient to ablate target tissue 182 through balloon 3120, while in other embodiments, the fluid may be sufficiently cold to freeze target tissue 182 through balloon 3120. The temperature of the fluid and duration for which the treatment is applied can be varied to control the type and depth of treatment.

Balloon catheter system 3100, in an embodiment, can further include an infusion device 10, as shown in FIGS. 1 and 3, for advancing fluid from the fluid source through inflow conduit 3112 and outflow conduit 3114. Infusion device 10 as it should be appreciated, may be any device suitable for advancing fluid from the fluid source through inflow conduit 3112 and outflow conduit 3114, such as a pump. In some embodiments, inflow conduit 3112 and outflow conduit 3114 may be sized such that fluid is advanced into and out of balloon 3120 at matching flow rates in order to keep the volume and pressure of balloon 3120 constant.

### Balloon Catheter System 3140

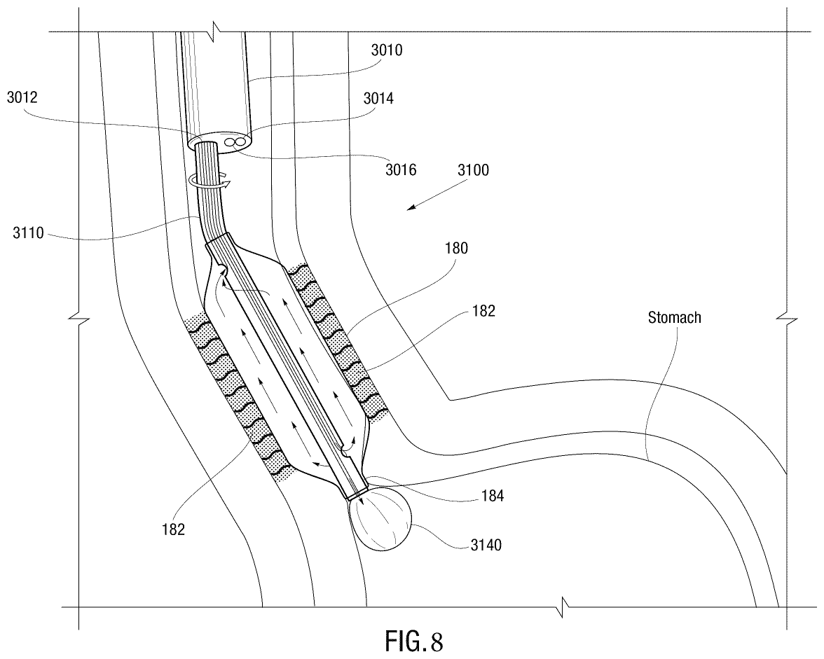

Referring now to FIGS. 8-11, in another representative embodiment, balloon catheter system 3100 may further include a balloon 3140 configured to help anchor system 3100 in place within body tract 180. It should be appreciated that there can be many forces that can cause balloon 3120 to move or migrate away from target issue 182 during treatment. For example, muscle contractions such as those associated with swallowing or gagging may push or pull on balloon 3120 and thereby move balloon 3120 up or down body tract 180. Likewise, an operator of system 3100 may inadvertently push or pull on assembly 3110 (i.e., elongate member), during treatment, causing balloon 3120 to move away from target tissue 182. This can be particularly problematic in embodiments in which the operator may need to rotate balloon 3120 to treat different areas about the circumference of body tract 180. Still further, balloon 3120 may often be advanced to the treatment site in a deflated state and subsequently inflated during treatment. As balloon 3120 expands, it may migrate away from target tissue 182, especially if that portion of tract 180 has a non-uniform diameter (e.g., narrows or widens).

In an effort to minimize the system 3100 from migrating within the treatment area, the system can include an additional expandable member 3140, i.e., a balloon, in various embodiments, which may be spaced apart from balloon 3120 lengthwise along elongate member 3110 such that balloon 3140 is positioned more distal than balloon 3120 on elongate member 3110 or more proximal than balloon 3120 on elongate member 3110. For example, in the representative embodiment shown in FIG. 8, balloon 3140 is positioned on a distal end of elongate member 3110.

Balloon 3140, in various embodiments, may be configured to help anchor balloon 3120 in place within body tract 180 by blocking advancement and/or retreat of elongate member 3110 and balloon 3120. In such a configuration, balloon 3140 may be configured to expand to dimensions greater than a diameter of a passageway 184 of body tract 180 such that, when positioned beyond passageway 184 and expanded, balloon 3140 cannot be withdrawn through passageway 184. Since balloon 3140 and balloon 3120 are fixedly connected by elongate member 3110, balloon 3120 may therefore be prevented from being withdrawn. Likewise, balloon 3140 can alternatively be positioned on a proximal side of passageway 184. In such a location, balloon 3140, when expanded, can also act as an anchor, and help to secure balloon 3120 and prevent it from being advanced within body tract 180.

By way of example, as shown in FIG. 8, balloon 3140 may be positioned beyond passageway 184, adjacent the gastroesophageal junction where the esophagus joins the stomach. At this location, there is a ring of muscles called the lower esophageal sphincter that controls movement of food from the esophagus into the stomach. Balloon 3140 may be positioned in the stomach beyond the lower esophageal sphincter and expanded within the stomach to dimensions greater than the diameter of the lower esophageal sphincter. As expanded, balloon 3140 cannot fit back through the lower esophageal sphincter and thus balloon 3140 can prevent elongate member 3110 and balloon 3120 from being withdrawn.

Still referring to FIG. 8, the lengthwise spacing between balloon 3120 and balloon 3140 may be such that balloon 3120 is positioned adjacent target tissue 182 when balloon 3140 is positioned beyond passageway 184, thereby ensuring proper alignment of each balloon within the anatomy. For example, when treating the lower esophagus, the lengthwise spacing between balloon 3120 and balloon 3140 may be relatively small since the lower esophagus is close to the gastroesophageal junction beyond which balloon 3140 is to be positioned for anchoring. Conversely, when treating the upper esophagus, the lengthwise spacing between balloon 3120 and balloon 3140 may be relatively large since the upper esophagus is farther away from the gastroesophageal junction beyond which balloon 3140 may be to be positioned for anchoring. For example, assuming the average person's esophagus is about 25 centimeters long, in various embodiments, the lengthwise spacing between balloon 3120 and balloon 3140 may be about 0-5 centimeters when treating the lower esophagus, about 5-15 centimeters when treating the mid esophagus, and about 15-25 centimeters when treating the upper esophagus. Of course, the spacing between balloon 3120 and balloon 3140 may vary based on the age and size of a particular patient and the specific area of the esophagus to be treated. One of ordinary skill in the art will recognize a suitable lengthwise spacing for a particular application in view of the teachings of the present disclosure and general knowledge regarding human anatomy.

Additionally, or alternatively, rather than blocking movement, balloon 3140 may be configured to expand against surrounding tissue to help anchor balloon 3120 in place within body tract 180. In such a configuration, pressure exerted by the expanded balloon 3140 against surrounding tissue generates corresponding normal and frictional forces that prevent balloon 3140 from being advanced or withdrawn along body tract 180. Since balloon 3140 and balloon 3120 are fixedly connected by elongate member 3110, balloon 3120 can therefore be prevented from being advanced or withdrawn.

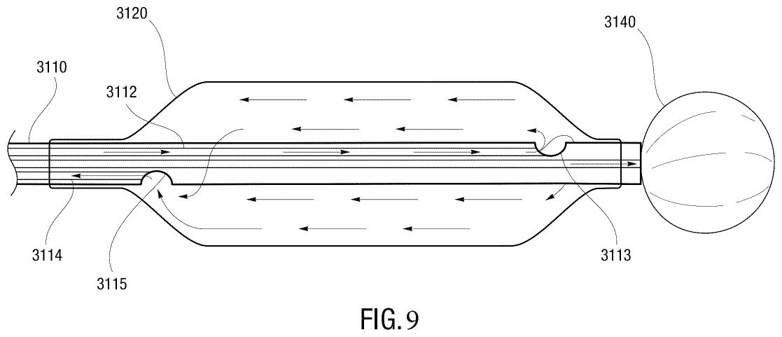

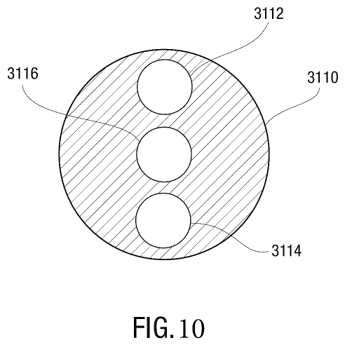

FIG. 9 shows a side view, and FIG. 10 depicts cross-sectional view, of a distal portion of system 3100 to show how balloon 3140 may be inflated/deflated. In particular, elongate member 3110 of the present embodiments may include an inflation/deflation conduit 3116 through which fluid can be directed into and out of balloon 3140 from a fluid source for expansion and deflation, respectively. Conduit 3116, in various embodiments, may be connected to a separate fluid source as conduit 3112, while in other embodiments, conduit 3112 and conduit 3116 may share a fluid source. In one embodiment, it may be desirable to use separate fluid sources, since filling balloon 3140 with the same hot or cold fluid used for treatment in balloon 3120 could result in unwanted application of thermal energy to non-target tissue surrounding balloon 3140. Of course, this may not be a concern if balloon 3140 is sufficiently thermally or if balloon 3140 is positioned in an area where such application of thermal energy is not problematic. Likewise, this may not be a concern if fluid being directed to balloon 3120 via conduit 3112 is separately heated or cooled downstream of the fluid source from that being directed to balloon 3140 via conduit 3116. As in the embodiments of FIGS. 4-7, in the present embodiments, fluid may be directed into balloon 3120 through inflow conduit 3112 and inflow port 3113, and out of balloon 3120 through an outflow conduit 3114 and outflow port 3115.

FIG. 11 depicts a front cross-sectional view of a distal portion of system 3100 showing balloon 3120 and balloon 3140 in deflated (labeled “D”) and inflated (labeled “I”) states. In various embodiments, balloon 3120 and balloon 3140 may be inserted into the body in a deflated state to make it easier to safely navigate balloon 3120 into position adjacent a target tissue 182 to be treated. Similar to the embodiments of FIGS. 4-7, elongate member 3110, balloon 3120, and balloon 3140 may be dimensioned such that these components can be advanced through lumen 3012 of endoscope 3010 when balloons 3120, 3140 are deflated and collapsed against elongate member 3110. Once positioned, in an embodiment, balloon 3140 may be expanded to anchor system 3100 in place within tract 180 and the balloon 3120 may be inflated by directing the fluid into and out of balloon 3120 through inflow and outflow conduits 3112, 3114, respectively. In this way, balloon 3120 can be ensured not to migrate away from target tissue 182 as balloon 3120 is expanded. In another embodiment, balloon 3120 may be expanded first, followed by balloon 3140. Such an approach may allow the operator to make adjustments in the placement of balloon 3120 prior to anchoring system 3100 in place. In yet another embodiment, balloon 3120 and balloon 3140 may be expanded simultaneously, or in an alternating fashion in which both balloons are partially expanded to confirm positioning before fully expanding each for treatment and anchoring, respectively.

Treatment may then proceed as previously described in the context of FIGS. 4-7. Upon completion of the treatment, balloon 3120 may be deflated by ceasing directing fluid into balloon 3120 and by withdrawing any remaining fluid from balloon 3120 if necessary, and balloon 3140 may be deflated by withdrawing fluid from balloon 3140 through inflation/deflation conduit 3116. Deflated balloons 3120, 3140 may then be withdrawn back through lumen 3012 of endoscope 3010 for removal from the body.

### Balloon Catheter System 3200

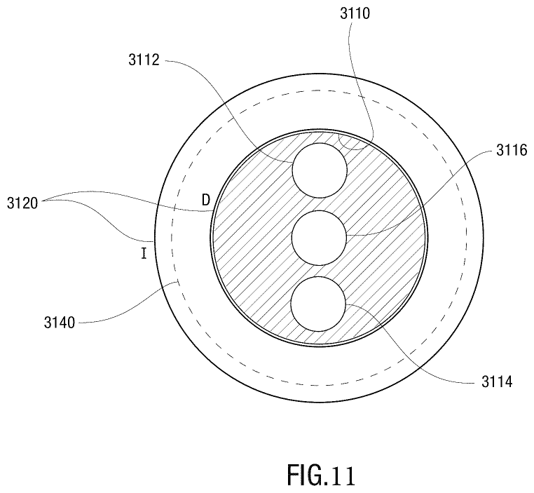

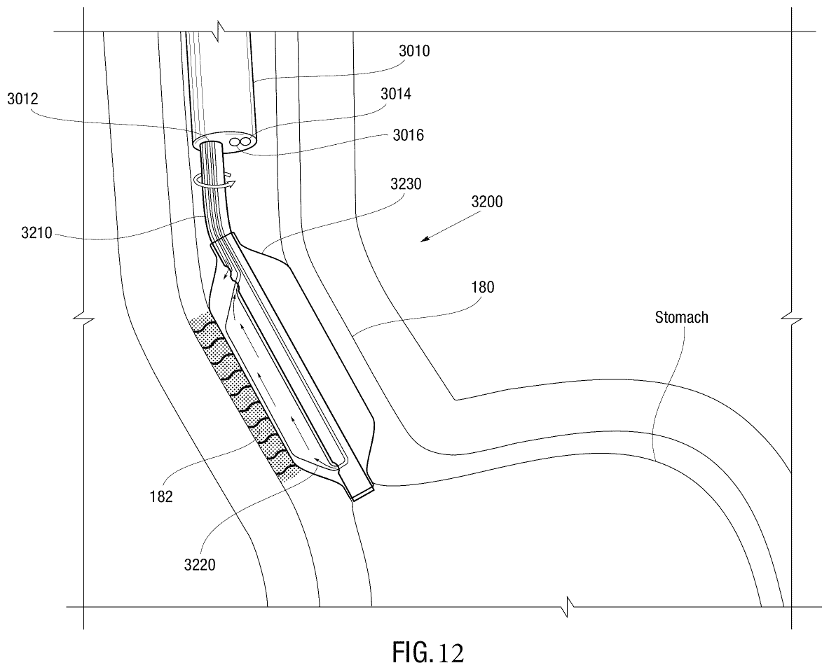

FIGS. 12-15 illustrate a representative embodiment of another alternative balloon catheter system 3200 for treating tissue within the body. System 3200, in various embodiments, may generally include an elongate member 3210, an inner balloon 3220 positioned near a distal end of elongate member 3210 and an outer balloon 3230 within which the inner balloon is situated. Except as set forth below, elongate member 3210 and inner balloon 3220 are generally analogous to elongate member 3110 and balloon 3120 and system 3200 may be used in similar fashion with endoscope system 3000 as previously described in the context of system 3100.

Outer balloon 3230, in various embodiments, may be configured to allow thermal energy from inner balloon 3220 to pass through outer balloon 3230 to treat surrounding target tissue 182. For example, the outer balloon 3230 may be made of a material with a low thermal resistance to permit thermal energy to readily pass therethrough from the inner balloon 3220 to the target tissue. In some embodiments, the outer balloon 3230 may be formed from polyurethane, nylon, polyethylene, PET or PEBAX, or any material that permits the outer balloon 3220 to be enlarged and permit for proper heat transfer between the inner balloon 3220 and the target tissue 182. In a preferred embodiment, outer balloon 3230 can be sufficiently thin and made of materials that provide minimal thermal insulation, or thermal resistance, which may otherwise degrade thermal energy transfer between the fluid being continuously directed into and out of inner balloon 3220 and target tissue 182. One of ordinary skill in the art will recognize without undue experimentation suitable thicknesses and materials of outer balloon 3230 that minimize, or otherwise allow for a desired level of, such thermal transfer between inner balloon 3220 and target tissue 182.

Outer balloon 3230, in various embodiments, may be coaxial with elongate member 3210. As configured, outer balloon 3230 may expand in radially about elongate member 3210 and thereby contact tissue substantially about the circumference of the associated portion of tract 180. Much like anchor balloon 3140 of system 3100, outer balloon 3230, in various embodiments, may be expanded against surrounding tissue to anchor system 3200 in place within tract 180. Additionally, or alternatively, outer balloon 3230, in various embodiments, may be expanded to create an enclosed space in which inner balloon 3220 can be freely rotated and/or translated without interference from surrounding tissue.

Inner balloon 3220, in various embodiments, may be smaller than outer balloon 3230 and offset from the axis shared by outer balloon 3230 and elongated member 3210, as shown in FIG. 12. As configured, inner balloon 3220 may expand outwards from elongate member 3210 to fill a partial azimuth of the volume defined by outer balloon 3230 such that inner balloon 3220 treats tissue along only a portion of the circumference of surrounding tract 180. For example, as shown in FIG. 12, while outer balloon 3230 expands in all radial directions about elongate member 3210 to contact the entire circumference of tract 180, offset inner balloon 3220 expands outwards from elongate member 3210 in one radial direction to treat a specific portion of the tissue surrounding outer balloon 3230—namely, a portion situated along the back side of the esophagus in this particular example.

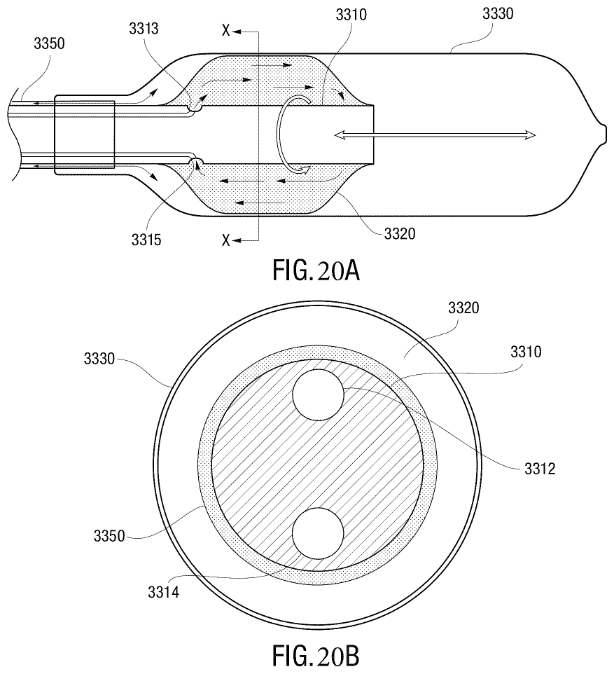

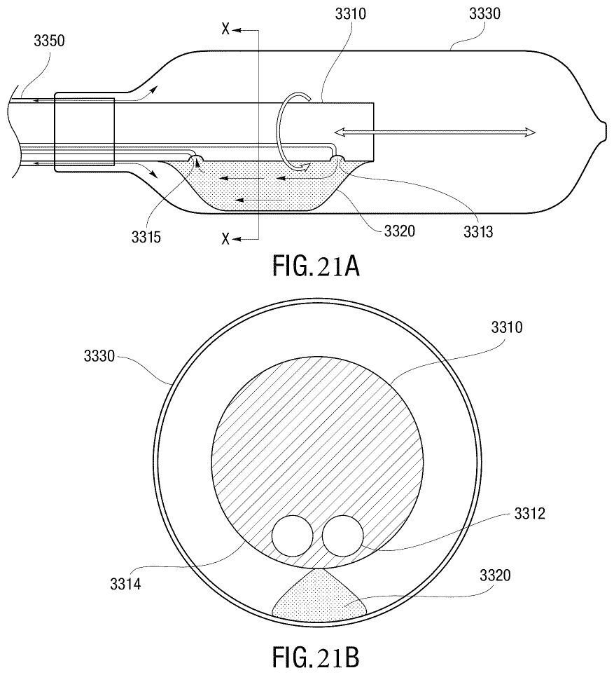

In various other embodiments (not shown), inner balloon 3220 and outer balloon 3230 may be coaxial with one another and with elongate member 3210. As configured, outer balloon 3230 and inner balloon 3220 may both expand outwards from elongate member 3210 in all radial directions to contact and treat surrounding tissue about the entire circumference of the associated portion of tract 180, respectively. In various embodiments, inner balloon 3220 and outer balloon 3230 are each fixedly coupled to elongate member 3210 such that rotating and translating elongate member 3210 serves to also rotate and translate inner balloon 3220 and outer balloon 3230 in unison. In such embodiments, an operator could treat multiple sections along a length of tract 180 by partially or fully deflating outer balloon 3230 to release any anchoring effect (if necessary) and translating the distal end of system 3200 along a length of tract 180 to position inner balloon 3220 and outer balloon 3230 adjacent another target tissue 182 of tract 180 to be treated. As described in more detail in the context of FIG. 20A, FIG. 20B, FIG. 21A, and FIG. 21B, in various embodiments, inner balloon 3220 may be configured to translate independent of outer balloon 3230. As configured, in an embodiment, outer balloon 3230 could remain in place within tract 180 and inner balloon 3220 (having a length shorter than that of outer balloon 3230) could be translated forward and/or backwards within outer balloon 3230 to treat specific portions of the tissue along an associated length of tract 180.

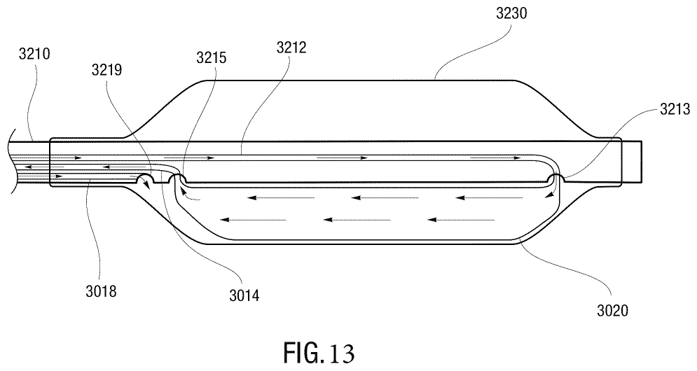

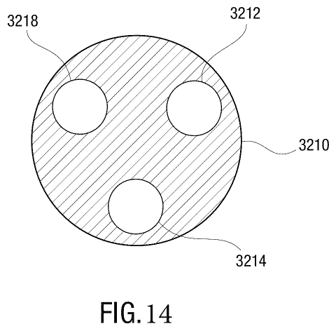

FIG. 13 and FIG. 14 depict side and front cross-sectional views respectively of a distal portion of system 3200. As shown in FIG. 13, fluid can be directed in and out of inner balloon 3220 in similar fashion as with balloon 3120. In particular, fluid may be directed into inner balloon 3220 through an inflow conduit 3212 and out of inner balloon 3220 through an outflow conduit 3214. Inflow conduit 3212 and outflow conduit 3214 may run within (as shown in FIG. 34) or along (not shown) elongate member 3210 and may be placed into fluid communication with an interior portion of inner balloon 3220 via inflow port 3213 and outflow port 3215, respectively. In the embodiment shown, inflow port 3213 can be situated near a distal end of inner balloon 3220 and outflow port 3215 can be situated near a proximal end of inner balloon 3220 such that fluid may be directed into and out of inner balloon 3220 from the distal end towards the proximal end. Of course, the locations of inflow port 3213 and outflow port 3215 may be reversed such that fluid can be directed into and out of inner balloon 3220 from the proximal end towards the distal end to similar effect. It should be recognized that by spacing out inflow port 3213 and outflow port 3215 to be located near opposing ends of inner balloon 3220, the fluid can be encouraged to circulate, or travel, through substantially the entire length of inner balloon 3220 and out via outflow port 3215, and thereby maintain a substantially uniform temperature distribution along the length of inner balloon 3220. This fluid circulation may make it easier to control the treatment since the operator would know that all tissue adjacent to inner balloon 3220 is receiving the same dose of thermal energy.

In various embodiments, inner balloon 3220 and outer balloon 3230 are each coupled to elongate member 3210 such that rotating and translating elongate member 3210 serves to also rotate and translate inner balloon 3220 and outer balloon 3230 in unison. In such embodiments, an operator could treat multiple target tissues 182 by partially or fully deflating outer balloon 3230 to release any anchoring effect (if necessary) and moving (e.g., translating and/or rotating) the distal end of system 3200 to position inner balloon 3220 adjacent another target tissue 182 of tract 180 to be treated. To the extent desired, inner balloon 3220, in an embodiment, can be designed to rotate and/or translate independent of outer balloon 3230. That way, outer balloon 3230 could remain in place within tract 180 while inner balloon 3220 rotated and/or translated therewithin to treat specific portions of the tissue about the circumference of tract 180.

As further shown in FIGS. 13 and 14, elongate member 3210 of the present embodiments may include an inflation/deflation conduit 3218 through which fluid can be directed into and out of outer balloon 3230 via port 3219 for expansion and deflation, respectively. Conduit 3218, in various embodiments, may be connected to a separate fluid source as conduit 3212, while in other embodiments, conduit 3212 and conduit 3218 may share a fluid source. In certain embodiments, it may be desirable to use separate fluid sources, since filling outer balloon 3230 with the same hot or cold fluid used for treatment in inner balloon 3220 could result in unwanted application of thermal energy to non-target tissue surrounding outer balloon 3230. This may not be a concern if fluid being directed to inner balloon 3220 via conduit 3212 is separately heated or cooled downstream of the fluid source from that being directed to outer balloon 3230 via conduit 3218.

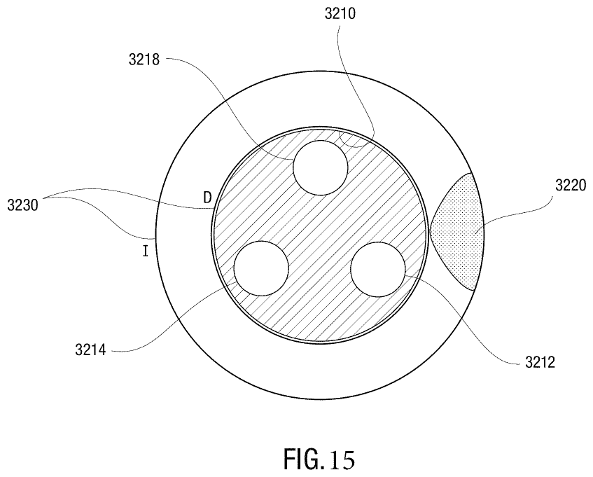

FIG. 15 depicts a front cross-sectional view of a distal portion of system 3200 showing inner balloon 3220 and outer balloon 3230 in deflated (labeled “D”) and inflated (labeled “I”) states. In various embodiments, the distal portion of system 3200 may be inserted into the body with inner balloon 3220 and outer balloon 3230 in a deflated state to make it easier to safely navigate inner balloon 3220 and outer balloon 3230 into position adjacent a target tissue 182 to be treated. Elongate member 3210, inner balloon 3220, and outer balloon 3230 may be dimensioned such that these components can be advanced through lumen 3012 of endoscope 3010 when balloons 3220, 3230 are deflated and collapsed against elongated member 3210. Once positioned, in an embodiment, outer balloon 3230 may be expanded via inflation/deflation conduit 3218 to anchor system 3200 in place within tract 180 and/or to create an enclosed space in which inner balloon 3220 can be freely rotated and/or translated without interference from surrounding tissue. Inner balloon 3220 may be inflated by directing the fluid into and out of inner balloon 3220 through inflow and outflow conduits 3212, 3214, respectively. This order can help ensure inner balloon 3220 does not migrate away from target tissue 182 as inner balloon 3220 is expanded. In another embodiment, inner balloon 3220 may be expanded first, followed by outer balloon 3230. Such an approach may allow the operator to make adjustments in the placement of inner balloon 3220 prior to anchoring system 3200 in place. In yet another embodiment, inner balloon 3220 and outer balloon 3230 may be expanded simultaneously, or in an alternating fashion in which both balloons are partially expanded to confirm positioning before fully expanding each for treatment and anchoring, respectively.

System 3200, in various embodiments, may further include at least one device (not shown) for advancing fluid from the fluid source through inflow conduit 3212 and outflow conduit 3214. In an embodiment, the device may comprise infusion device 10 disclosed herein. In another embodiment, the device may comprise any other device suitable for advancing fluid from the fluid source through inflow conduit 3212 and outflow conduit 3214, such as a pump. In some embodiments, inflow conduit 3212 and outflow conduit 3214 may be sized such that fluid is advanced into and out of balloon 3220 at matching flow rates in order to keep the volume and pressure of balloon 3220 constant.

Like system 3100, system 3200 may be configured to treat a variety of portions of the body and ailments such as, without limitation, treating tissue in the upper gastrointestinal tract, treating tissue in the lower gastrointestinal tract, ablating parasympathetic nerves in the renal artery, ablating parasympathetic nerves in the nasal cavity, ablating vessels in the circulatory systems, ablating fistulae tracts, and treating breast tissue.

### Balloon Catheter System 3200

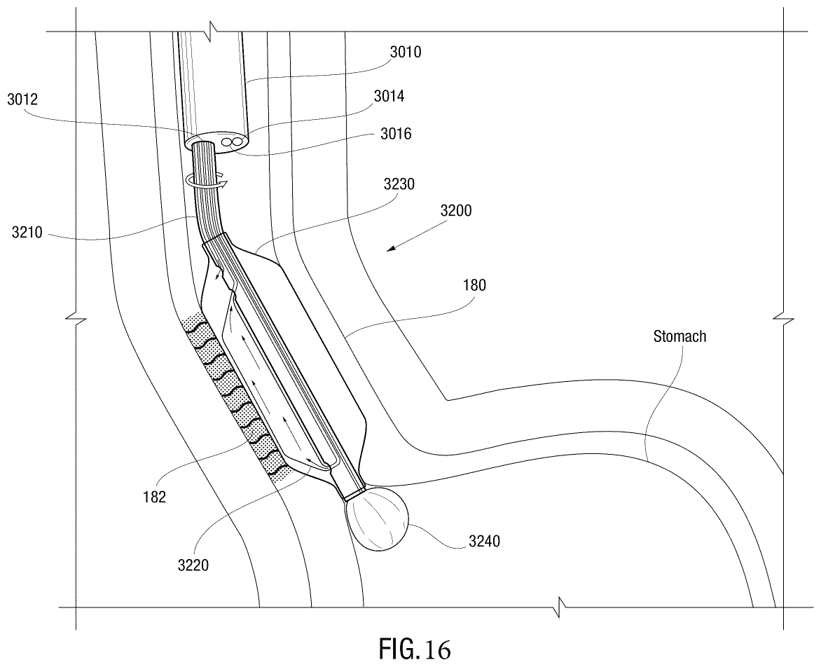

Referring now to FIGS. 16-19, in another representative embodiment, system 3200 may further include a balloon 3240 configured to help anchor system 3200 in place within body tract 180. Balloon 3240, in various embodiments, may be spaced apart from outer balloon 3230 lengthwise along elongated member 3210 such that balloon 3240 may be positioned more distal than outer balloon 3230 on elongated member 3210 or more proximal than outer balloon 3230 on elongated member 3210. For example, in the representative embodiment shown in FIG. 16, balloon 3240 can be positioned on a distal end of elongate member 3210.

Balloon 3240, as in various embodiments, may be configured to help, or assist, to anchor outer balloon 3230 in place within body tract 180 by blocking advancement and/or retreat of elongate member 3210 and outer balloon 3230. In such a configuration, balloon 3240 may be configured to expand to dimensions greater than a diameter of a passageway 184 of body tract 180 such that, when positioned beyond passageway 184 and expanded, balloon 3240 cannot be withdrawn through passageway 184. In some embodiments, balloon 3240 and outer balloon 3230 can be fixedly connected together via the elongate member 3110 such that outer balloon 3230 may therefore be prevented from being withdrawn as well. Likewise, when balloon 3240 is positioned on a proximal side of passageway 184 and expanded, balloon 3240 cannot be advanced through passageway and thus outer balloon 3230 is prevented from being advanced within body tract 180 as well.

By way of example, as shown in FIG. 16, balloon 3240 may be positioned beyond passageway 184, here the gastroesophageal junction where the esophagus joins the stomach. At this location, there is a ring of muscles called the lower esophageal sphincter that controls movement of food from the esophagus into the stomach. Balloon 3240 may be positioned in the stomach beyond the lower esophageal sphincter and expanded within the stomach to dimensions greater than the diameter of the lower esophageal sphincter. As expanded, balloon 3240 cannot fit back through the lower esophageal sphincter and thus balloon 3240 prevents elongate member 3210 and outer balloon 3230 from being withdrawn as well.

Still referring to FIG. 16, the lengthwise spacing between outer balloon 3230 and balloon 3240 may be such that outer balloon 3230 (and thus inner balloon 3220) is positioned adjacent target tissue 182 when balloon 3240 is positioned beyond passageway 184, thereby ensuring proper alignment of each balloon within the anatomy. For example, when treating the lower esophagus, the lengthwise spacing between outer balloon 3230 and balloon 3240 may be relatively small since the lower esophagus is close to the gastroesophageal junction beyond which balloon 3240 is to be positioned for anchoring. Conversely, when treating the upper esophagus, the lengthwise spacing between outer balloon 3230 and balloon 3240 may be relatively large since the upper esophagus is farther away from the gastroesophageal junction beyond which balloon 3240 is to be positioned for anchoring. For example, assuming the average person's esophagus is about 25 centimeters long, in various embodiments, the lengthwise spacing between balloon 3230 and balloon 3240 may be about 0-5 centimeters when treating the lower esophagus, about 5-15 centimeters when treating the mid esophagus, and about 15-25 centimeters when treating the upper esophagus. Of course, the spacing between balloon 3120 and balloon 3140 may vary based on the age and size of a particular patient and the specific area of the esophagus to be treated. One of ordinary skill in the art will recognize a suitable lengthwise spacing for a particular application in view of the teachings of the present disclosure and general knowledge regarding human anatomy.

Additionally, or alternatively, rather than blocking movement, balloon 3240 may be configured to expand against surrounding tissue to help anchor the distal portion of system 3200 in place within body tract 180. In such a configuration, pressure exerted by the expanded balloon 3240 against surrounding tissue can generate corresponding normal and frictional forces that prevent balloon 3240 from being advanced or withdrawn along body tract 180. Since balloon 3240 and outer balloon 3230 are fixedly connected by elongate member 3210, outer balloon 3230 is therefore is prevented from being advanced or withdrawn as well.

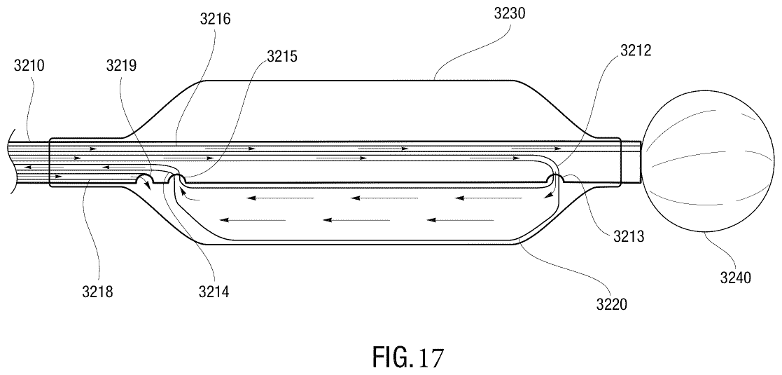

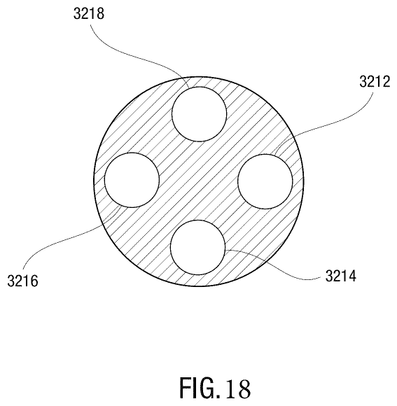

FIG. 17 and FIG. 18 depict side and front cross-sectional views of a distal portion of system 3200 to show how balloon 3240 can be inflated/deflated. In particular, elongate member 3210 of the present embodiments may include an inflation/deflation conduit 3216 through which fluid can be directed into and out of balloon 3240 for expansion and deflation, respectively. Conduit 3216, in various embodiments, may be connected to a separate fluid source as conduit 3212 and/or conduit 3218, while in other embodiments, conduit 3212, conduit 3218, and/or conduit 3116 may share a fluid source. In some embodiments, as shown in FIG. 18, the various conduits 3213, 3214, 3216, 3218 are shown as extending in parallel within the elongate member 3210. Alternatively, the various conduits 3213, 3214, 3216, 3218 can be “nested” such that they are concentric with one another. In certain embodiments, it may be desirable to use separate fluid sources, since filling balloon 3240 with the same hot or cold fluid used for treatment in inner balloon 3220 could result in unwanted application of thermal energy to non-target tissue surrounding balloon 3240. Of course, this may not be a concern if balloon 3240 is sufficiently thermally or if balloon 3240 is positioned in an area where such application of thermal energy is not problematic. Likewise, this may not be a concern if fluid being directed to inner balloon 3220 via conduit 3212 is separately heated or cooled downstream of the fluid source from that being directed to balloon 3240 via conduit 3216. Fluid may be directed into inner balloon 3220 through inflow conduit 3212 and inflow port 3213, and out of inner balloon 3220 through an outflow conduit 3214 and outflow port 3215.

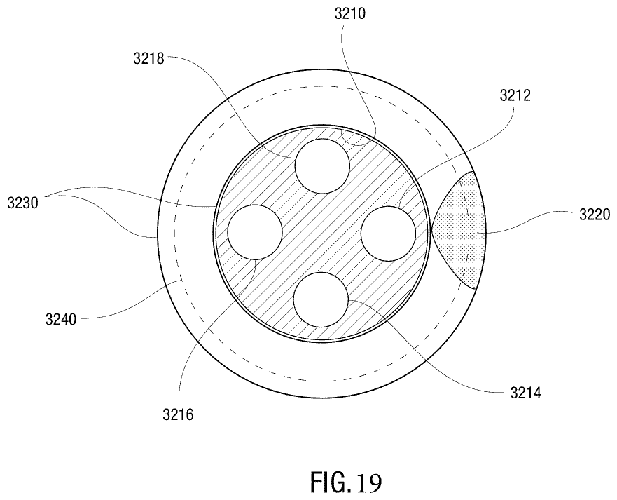

FIG. 19 depicts a front cross-sectional view of a distal portion of system 3200 showing outer balloon 3230 in deflated (labeled “D”) and inflated (labeled “I”) states, and inner balloon 3220 and anchor balloon 3240 in inflated states. In various embodiments, the distal portion of system 3200 may be inserted into the body with balloons 3220, 3230, and 3240 in a deflated state to make it easier to safely navigate outer balloon 3230 into position adjacent a target tissue 182 to be treated. Elongate member 3210 and balloons 3220, 3230, and 3240 may be dimensioned such that these components can be advanced through lumen 3012 of endoscope 3010 when balloons 3220, 3230, and 3240 are deflated and collapsed against elongated member 3210. Once positioned, in an embodiment, balloons 3230, 3240 may be expanded to anchor system 3200 in place within tract 180 and inner balloon 3220 may be inflated by directing the fluid into and out of inner balloon 3220 through inflow and outflow conduits 3212, 3214, respectively. This order can help ensure inner balloon 3220 does not migrate away from target tissue 182 as inner balloon 3220 is expanded. In another embodiment, inner balloon 3220 may be expanded first, followed by balloons 3230, 3240. Such an approach may allow the operator to make adjustments in the placement of inner balloon 3220 prior to anchoring system 3200 in place. In yet another embodiment, balloons 3220, 3230, and 3240 may be expanded simultaneously, or in an alternating fashion in which both balloons are partially expanded to confirm positioning before fully expanding each for treatment and anchoring, respectively.

Treatment may then proceed as previously described. Upon completion of the treatment, inner balloon 3220 may be deflated by ceasing directing fluid into inner balloon 3220 and by withdrawing any remaining fluid from balloons 3220 if necessary, and balloons 3230, 3240 may be deflated by withdrawing fluid from balloons 3230, 3240 through inflation/deflation conduits 3218, 3216, respectively. Deflated balloons 3220, 3230, and 3240 may then be withdrawn back through lumen 3012 of endoscope 3010 for removal from the body.

### Balloon Catheter System 3300

FIGS. 20A, 20B, 21A, and 21B illustrate two representative embodiments of other alternative balloon catheter systems 3300 for treating tissue within the body. System 3300, in various embodiments, may generally include an elongate member 3310, an inner balloon 3320, an outer balloon 3330, a sheath 3350, and endoscope system 3000. Except as set forth below, elongate member 3310, inner balloon 3320, and outer balloon 3330 are generally analogous to elongate member 3210, inner balloon 3220, and outer balloon 3230 and system 3300 may be used in similar fashion with endoscope system 3000 as previously described in the context of systems 3100 and 3200.

System 3300, in various embodiments, may be configured to allow inner balloon 3320 to rotate and translate within outer balloon 3330. In particular, elongate member 3310 may positioned within a substantially hollow sheath 3350 (e.g., a catheter) and dimensioned such that elongate member 3310 can rotate as well as slide proximally and distally within sheath 3350. As shown, the proximate end of outer balloon 3330 may be coupled to the distal end of sheath 3350 and inflated/deflated through conduit 3318 defined by the open space between the outer diameter of elongate member 3310 and inner diameter of sheath 3350. As configured, outer balloon 3330 could be anchored in place within tract 180 and inner balloon 3320 (having, in an embodiment, a length shorter than that of outer balloon 3330) could be translated proximally and/or distally within outer balloon 3330 to treat specific portions of the tissue along an associated length of tract 180. Additionally, or alternatively, the inner balloon 3320 could rotate about a central axis within, and relative to, the outer balloon 3330. For example, certain portions of the tissue may require additional thermal treatment while other portions of tissue may require less thermal treatment. Advantageously, system 3300 allows for the inner balloon 3320 to translate within the outer balloon 3330, without having to deflate and re-inflate the outer balloon 3330 to treat a different area of tissue.

FIGS. 20A and 20B depict an embodiment of system 3300 in which inner balloon 3320 can expand in all radial directions about elongate member 3310 to treat the entire circumference of tract 180 (much like balloon 3120), while FIGS. 21A and 21B depict another embodiment of system 3300 in which inner balloon 3220 can expand outwards from elongate member 3310 in one radial direction to treat a specific portion of the tissue surrounding outer balloon 3330 (much like the embodiment of balloon 3220 shown in FIG. 12). In both embodiments, inner balloon 3320 can be shorter than outer balloon 3330 to allow room for moving inner balloon 3320 forwards and backwards within outer balloon 3330 by virtue of advancing and retracting elongate member 3310 within sheath 3350. It should be recognized that the shorter inner balloon 3320 is relative to outer balloon 3330, the farther inner balloon 3320 can travel within the space defined by outer balloon 3330, and vice versa, to provide added resolution and specificity to the treatment area. In an embodiment, an operator may position outer balloon 3330 at an area to be treated, inflate outer balloon 3330 to anchor the proximal end of system 3300 and/or define an open space within which to freely move inner balloon 3320, and then rotate and/or translate inner balloon 3320 as needed to treat various places along the length of the treatment site.

Fluid can be directed in and out of inner balloon 3320 in similar fashion as with balloon 3220. In particular, fluid may be directed into inner balloon 3320 through an inflow conduit 3312 and out of inner balloon 3320 through an outflow conduit 3314. Inflow conduit 3312 and outflow conduit 3314 may run within (as shown) or along (not shown) elongate member 3310 (space permitting within conduit 3318) and may be placed into fluid communication with an interior portion of inner balloon 3320 via inflow port 3313 and outflow port 3315, respectively. In the embodiment shown, both inflow port 3313 and outflow port 3315 are situated near a proximal end of inner balloon 3320 and provide for directing fluid into and out of balloon 3320. Of course, the locations of inflow port 3313 and outflow port 3315 may be as shown and described in the context of system 3200 (with inflow port 3313 located near a distal end of balloon 3320 and outflow port 3315 located near a proximal end of balloon 3320) or reversed such that fluid can be directed into and out of inner balloon 3320 from the proximal end towards the distal end to similar effect. In both embodiments, the fluid can be described as being circulated, or driven, along substantially the entire length of the inner balloon 3320, as shown via the fluid flow lines within the inner balloon. The aforementioned fluid flow provides for the advantages described throughout the disclosure.

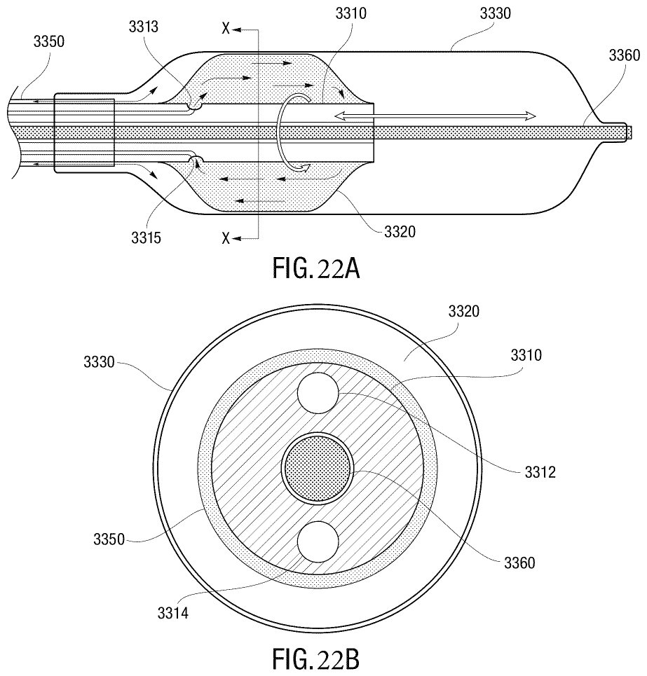

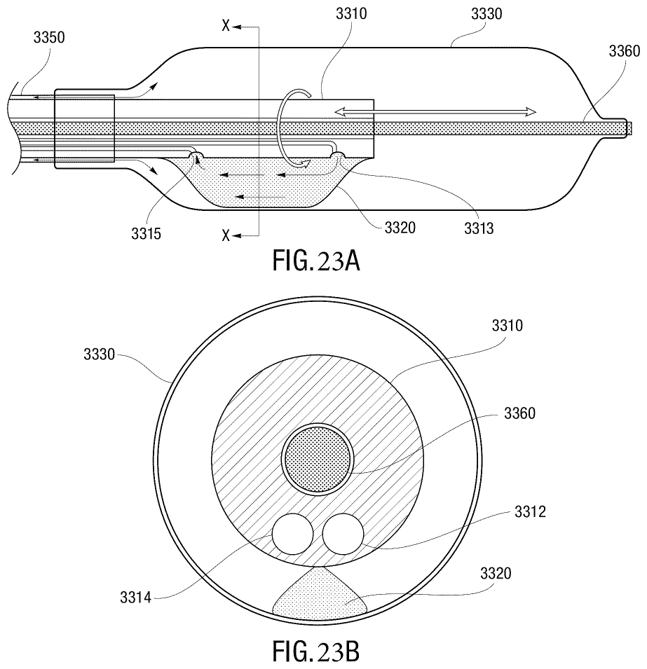

In a further alternative embodiment, referring now to FIGS. 22A, 22B, 23A, and 23B, system 3300 may further comprise an elongate member 3360. The embodiment of FIGS. 22A and 22B are largely analogous to the embodiment of FIGS. 20A and 20B, and the embodiment of FIGS. 23A and 23B are largely analogous to the embodiment of FIGS. 21A and 21B. A distal end of outer balloon 3330 may couple to a distal end of elongate member 3360 and elongate member 3310 may comprise a hollow portion configured to accommodate elongate member 3360 therethrough. As configured, elongate member 3310 may still slide proximally and distally along elongate member 3360 within sheath 3350. In various embodiments, elongate member 3360 may serve any one or combination of purposes including, for example, providing added stability for the longitudinal or rotational movement of the inner balloon 3320 within the outer balloon 3330, adding longitudinal rigidity to outer balloon 3330, providing a conduit for a guidewire along which the distal portion of system 3300 may be advanced and retracted, and/or providing a conduit for inflating and deflating a distal anchor balloon, if equipped (similar to conduits 3116, 3216 for inflating and deflating anchor balloons 3140, 3240).

### Balloon Catheter System 3400

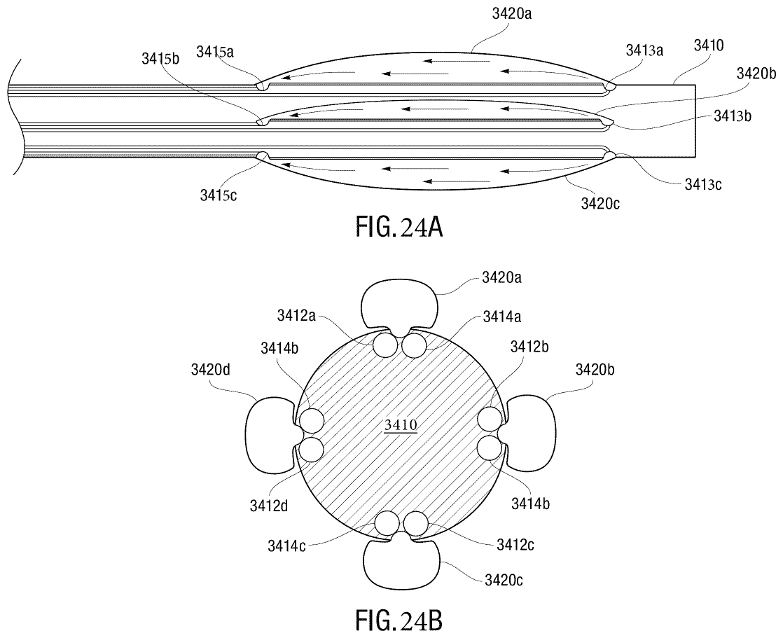

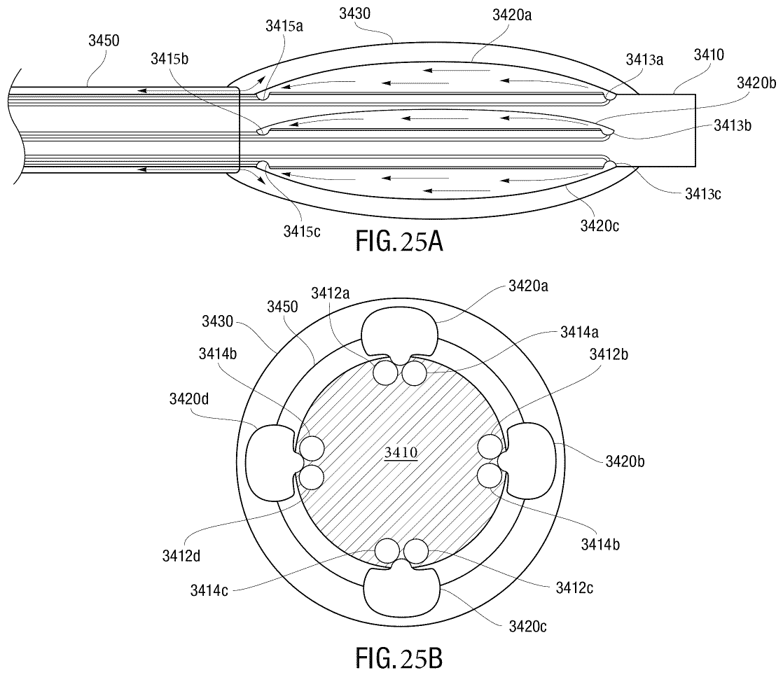

FIGS. 24A, 24B, 25A, and 25B illustrate representative embodiments of another balloon catheter system 3400 for treating tissue within the body. System 3400, in various embodiments, may generally include an elongate member 3410, two or more balloons 3420 positioned about a circumference of elongate member 3410, and endoscope system 3000. Except as set forth below, elongate member 3310 is generally analogous to elongate members 3110, 3210 and system 3400 may be used in similar fashion with endoscope system 3000 as previously described in the context of systems 3100, 3200, 3300.

Referring first to FIGS. 24A and 24B, system 3400 is shown with four balloons 3420a, 3420b, 3420c, 3420d circumferentially spaced apart equally about the circumference of elongate member 3410. Similar to balloons 3220 and 3320, each balloon 3420a, 3420b, 3420c, 3420d may expand outwards in a respective one radial direction to treat a specific portion of the surrounding tissue. In various embodiments, any number of balloons 3420a-d may be positioned about the circumference of elongate member 3410, and balloons 3420a-d may be positioned in any suitable arrangement (e.g., at equally spaced intervals or non-equally spaced intervals) about the circumference of elongate member 3410. For example, in some embodiments (not shown), balloons 3420a-d may be positioned on only one half or one quarter of elongate member 3410. Such configurations may be particularly suitable for precision treatment of tissue on one half or one quarter of the surrounding portion of tract 180. Additionally, or alternatively, system 3400 may be configured such that an operator may select which particular balloon(s) 3420 to use for treatment at any given time, thereby providing similar capabilities for precision treatment of specific tissue areas.

As shown in FIG. 24A, fluid may be directed in and out of each balloon 3420 in similar fashion as with balloons 3120, 3220, 3320. In particular, fluid may be directed into each balloon 3420 (e.g., 3420a, 3420b, 3420c, 3420d) through a respective inflow conduit 3412 (e.g., 3412a, 3412b, 3412c, 3412d) and out of each balloon 3420 through a respective outflow conduit 3414 (e.g., 3414a, 3414b, 3414c, 3414d). Inflow conduits 3412 and outflow conduits 3414 may run within (as shown in FIG. 24A, 24B) or along (not shown) elongate member 3410 and may each be placed into fluid communication with an interior portion of a corresponding balloon 3420 via an inflow port 3413 (e.g., 3413a, 3413b, 3413c, 3413d) and an outflow port 3415 (e.g., 3415a, 3415b, 3415c, 3415d), respectively. In the embodiment shown, inflow ports 3413 are situated near the distal ends of balloons 3420 and outflow ports 3315 are situated near the proximal ends of balloons 3420 such that fluid is directed into and out of each balloon 3420 from the distal end towards the proximal end. Of course, the locations of inflow ports 3413 and outflow ports 3315 may be reversed such that fluid is directed into and out of balloons 3420 from the proximal end towards the distal end to similar effect. It should be recognized that by spacing out each inflow port 3413 and each outflow port 3415 to be located near opposing ends of a corresponding balloon 3420, the fluid is encouraged to travel through the length of the balloon 3420 and out via outflow port 3415, and thereby maintain a substantially uniform temperature distribution along the length of the balloon 3420. This may make it easier to control the treatment since the operator would know that all tissue adjacent to a particular balloon 3420 is receiving the same dose of thermal energy. Conversely, were one or both of inflow port 3413 and outflow port 3415 located more towards the center of balloon 3420, fluid may stagnate and cool near opposing ends of balloon 3420, resulting in a substantially non-uniform temperature distribution along the length of balloon 3420. This may make it more difficult to control the treatment since tissue adjacent to inner balloon 3420 would, in turn, receive varied doses of thermal energy. Additionally, or alternatively, the fluid can be described as being circulated, or driven, along substantially the entire length of the respective balloon 3420a, 3420b, 3420c, 3420d, as shown via the fluid flow lines within the inner balloon. The aforementioned fluid flow provides for the advantages described throughout the disclosure.

Referring now to FIGS. 25A and 25B, in various embodiments, system 3400 may further comprise an outer balloon 3430 surrounding inner balloons 3420, similar to outer balloon 3230, 3330. Outer balloon 3430, in various embodiments, may be configured to allow thermal energy emitted from balloons 3420 to pass through outer balloon 3430 to treat surrounding target tissue 182. In a preferred embodiment, outer balloon 3430 is sufficiently thin and made of materials that provide minimal thermal insulation that may otherwise degrade thermal energy transfer between the fluid being continuously directed into and out of balloons 3420 and target tissue 182. One of ordinary skill in the art will recognize without undue experimentation suitable thicknesses and materials of outer balloon 3430 that minimize, or otherwise allow for a desired level of, such thermal transfer between balloons 3420 and target tissue 182.

Outer balloon 3430, in various embodiments, may be coaxial with elongate member 3410. As configured, outer balloon 3430 may expand in all radial directions about elongate member 3410 and thereby contact tissue about the entire circumference of the associated portion of tract 180. Much like anchor balloon 3140, 3240, outer balloon 3430, in various embodiments, may be expanded against surrounding tissue to anchor system 3400 in place within tract 180. Additionally, or alternatively, outer balloon 3430, in various embodiments, may be expanded to create an enclosed space in which balloons 3420 can be freely rotated and/or translated without interference from surrounding tissue.

### Concentric Conduit System 3600

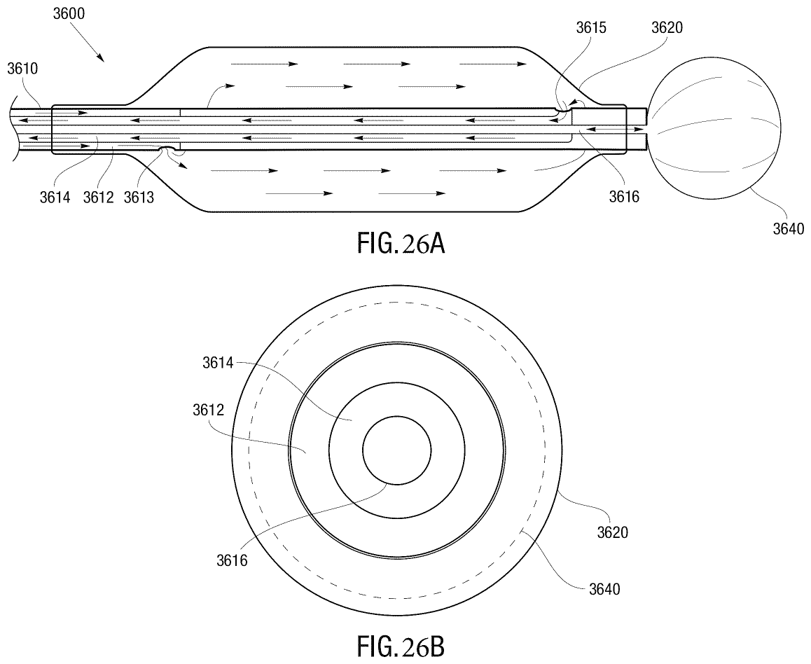

FIGS. 26A and 26B illustrate a representative embodiment of balloon catheter system 3600 in which the one or more conduits of an assembly (for example, assembly 20 in FIG. 1) are arranged in a concentric manner. System 3600, in various embodiments, may generally include an elongate member 3610, a treatment balloon 3620, and a distal anchor balloon 3640. Except as set forth below, elongate member 3310, treatment balloon 3320, and distal anchor balloon 3640 are generally analogous to elongate member 3110, balloon 3120, and distal anchor balloon 3140, and system 3600 may be used in similar fashion with an endoscope, e.g., endoscope system 3000, or any other endoscope, as previously described in the context of systems 3100 and 3200.

Elongate member 3610, in various embodiments, may include or support an assembly of one or more conduits, or fluid pathways, (e.g., conduits 3612, 3614, 3616) running along its length through which fluid(s) may be directed into and out of balloon 3620 and any additional balloons. As shown throughout the present figures, these conduits may be internal to elongate member 3610 and arranged in a concentric fashion. In the embodiment shown, inflow conduit 3612 can be the outermost conduit, followed by outflow conduit 3614 and anchor inflation/deflation conduit 3616 moving towards the center. In various embodiments, fluid may be directed into balloon 3620 through an inflow conduit 3612 from a fluid source and out of balloon 3620 through an outflow conduit 3614. Inflow conduit 3612 and outflow conduit 3614 may be placed into fluid communication with an interior portion of balloon 3620 via inflow port 3613 and outflow port 3615, respectively. In the embodiment shown, outflow port 3615 can be situated near a distal end of balloon 3620 and inflow port 3113 can be situated near a proximal end of balloon 3620 such that fluid can be directed into and out of balloon 3620 from the proximal end towards the distal end. Of course, the locations of inflow port 3613 and outflow port 3615 may be reversed such that fluid is directed into and out of balloon 3620 from the distal end towards the proximal end to similar effect. It should be recognized that by spacing out inflow port 3613 and outflow port 3615 to be located near opposing ends of balloon 3620, the fluid is encouraged to travel through the length of balloon 3620 and thereby maintain a substantially uniform temperature distribution along the length of balloon 3620. This may make it easier to control the treatment since the operator would know that all tissue adjacent to balloon 3620 is receiving the same dose of thermal energy. Conversely, were one or both of inflow port 3613 and outflow port 3615 located more towards the center of balloon 3620, fluid may stagnate and cool near opposing ends of balloon 3620, resulting in a substantially non-uniform temperature distribution along the length of balloon 3620. This may make it more difficult to control the treatment since tissue adjacent to balloon 3620 would, in turn, receive varied doses of thermal energy.

### Facilitating Advancement Through an Endoscope and the Body

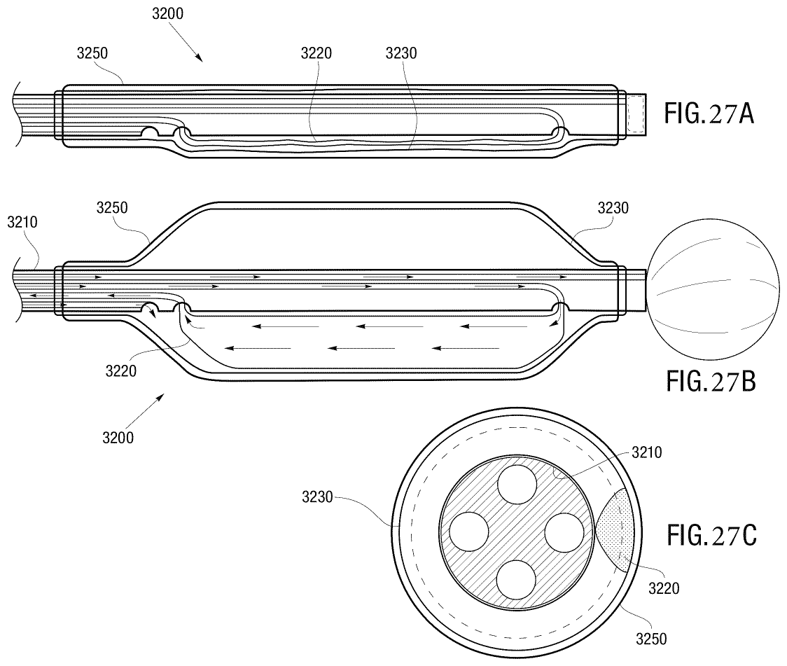

FIGS. 27A-27C, FIGS. 28A-28B, and FIGS. 29A-29C illustrate representative techniques for streamlining a profile of balloon systems of the present disclosure to facilitate advancement to the treatment site in tract 180. Generally speaking, the profile, or outer dimensions, of the various balloon systems may be minimized by collapsing and securing one or more of the balloons against the catheter member(s) prior to insertion into an endoscope 3010 or the body (and in some cases, at the manufacturer). As configured, in various embodiments, balloon systems may fit within lumen 3012 of endoscope 3010 and as such, can be advanced to the treatment site along with endoscope 3010 or subsequently directed to the treatment site through an endoscope 3010 already positioned within the body near the treatment site. Likewise, the streamlined profile of these balloon systems may allow them to be inserted through other instruments or more easily directed through the tract 180 on its own. While the following embodiments may be described in the context of a particular balloon system, it should be recognized that these embodiments can be adapted for use with other balloon systems without departing from the scope of the present disclosure.

FIGS. 27A, 27B, and 27C illustrate a representative embodiment of system 3200 in which a stent 3250 can surround the outer balloon 3230 and inner balloon 3220. Stent 3250, in various embodiments, may have elastic properties and may be dimensioned such that it collapses tightly about outer balloon 3230 and inner balloon 3220 when deflated. As configured, stent 3250 may hold deflated balloons 3220, 3230 tight against the outer surface of elongate member 3210 and thereby reduce an overall diameter of the distal end of balloon catheter 3200, as well as protect deflated balloons 3220, 3230 from damage. Due to its elastic properties, stent 3250 may expand as balloons 3220, 3230 are inflated without being removed. In various embodiments, the particular dimensions, and elastic properties of stent 3250 may be configured to allow stent 3250 to expand far enough to accommodate balloons 3220, 3230 in a fully inflated state and as such, not interfere with operation of balloon system 3200 in accordance with the disclosure herein. Stent 3250, in various embodiments, may be made from a material configured to both withstand the heat or cold applied by the treatment fluid within balloon 3220 without being damaged, as well as to not thermally insulate or otherwise substantially interfere with heat transfer between the fluid of balloon 3220 and target tissue 182 being treated. The material and design of stent 3250, in various embodiments, may be also be configured such that stent 3250 retains its elastic properties after treatment, so as to again collapse about balloons 3220, 3230 as they are deflated and thereby facilitate removal of balloon catheter system 3200 from the body (which, in some cases, may be back through lumen 3012 of endoscope 3010).

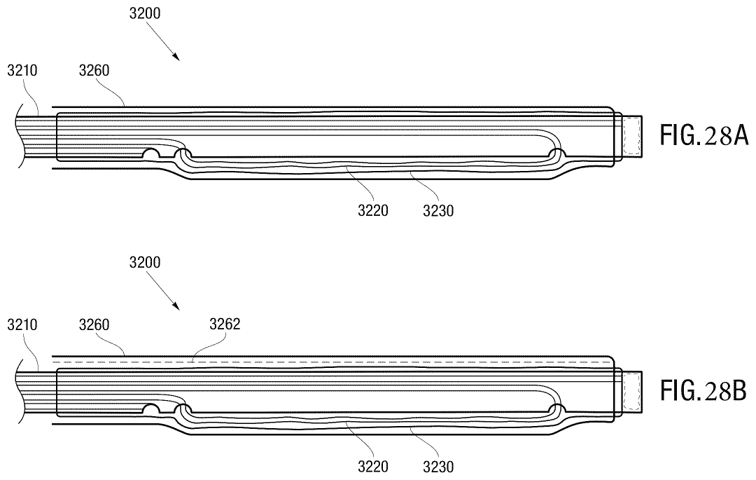

FIGS. 28A and 28B illustrate another representative embodiment of system 3200 in which a sleeve 3260 can surround the outer balloon 3230 and inner balloon 3220. Unlike stent 3250, in various embodiments sleeve 3260 may be configured to be removed from the distal end of system 3200 prior to treating the target tissue 182. Because sleeve 3260 may be removed from system 3200 within the body, sleeve 3260 may be made of a digestible, biodegradable, passable, or other material that will not harm the patient if left in the body after the treatment procedure, for example a gelatin material. Generally speaking, for procedures in the digestive system such as esophageal treatments, sleeve 3260 may be made of a digestible or passable material (e.g., gelatin) since sleeve 3260 will be left behind in the digestive system. Sleeve 3260 may be dimensioned to compress about or otherwise hold deflated balloons 3220, 3230 tight against the outer surface of elongate member 3210 and thereby reduce an overall diameter of the distal end of balloon catheter 3200, as well as protect deflated balloons 3220, 3230 from damage. Unlike stent 3250, sleeve 3260 may be configured to break, slide off of, or otherwise be removed from the distal end of system 3200 prior to treating the target tissue 182. In an embodiment, this may be accomplished by partially inflating balloon 3230 until the pressure it exerts against sleeve 3260 breaks sleeve 3260. Additionally, or alternatively, in another embodiment, hot or cold treatment fluid may be introduced into balloon 3220 to help break down sleeve 3260 for removal. As shown in FIG. 28B, sleeve 3260 may further comprise a perforation 3262 or other localized weakness to help ensure sleeve 3260 breaks away when balloons 3220, 3230 are inflated.

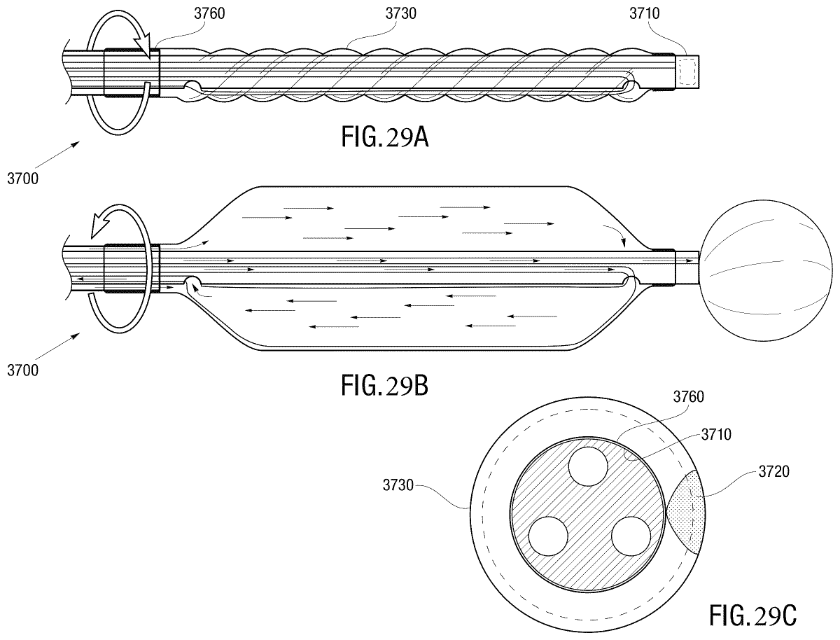

FIGS. 29A, 29B, and 29C illustrate a representative embodiment of system 3700 in which deflated balloon 3730 is twisted about elongate member 3710 to reduce the profile of the distal end of system 3700. In system 3700, a distal end of balloon 3730 may be coupled to a distal end of elongate member 3710, and a proximal end of balloon 3730 may be coupled to a distal end of elongate member 3760 and, as such, the ends of balloon 3730 are attached to different elongate members 3710, 3760. Similar to inner balloon 3220 of FIG. 27C, system 3700 can additionally include an inner balloon 3720, as well. In various embodiments, elongate member 3710 and elongate member 3760 may be rotatable relative to one another. As configured, holding elongate member 3710 in place while rotating elongate member 3760 (or vice versa) or, alternatively, rotating elongate member 3710 and elongate member 3760 in opposing directions, may cause balloon 3720 to twist about and thereby collapse on elongate member 3710. In some embodiments, system 3700 may come pre-twisted from the manufacturer while, in other embodiments, a user may perform the twisting technique prior to advancing system 3700 to the treatment site.

Elongate members 3710, 3760, in various embodiments, may be held in place to maintain deflated balloon 3720 in this twisted, collapsed state. In an embodiment, elongate members 3710, 3760 may be held together by glue, a rubber band, or some other temporary coupler whose bond can be broken or removed by the user. For example, elongate members 3710, 3760 may be glued together by the manufacturer and configured such that a user may break the glue bond by rotating elongate members 3710, 3760 in opposing directions with sufficient force to break the bond. In another embodiment, the proximal ends of elongate members 3710, 3760 may be coupled to a mechanism configured to prevent or resist rotation of elongate members 3710, 3760. The mechanism may be configured to allow a user to untwist balloon 3730 by rotating one of elongate members 3710, 3760 in a reverse direction as that originally used to twist balloon 3730. Detents or other resistive features may serve to hold elongate members 3710, 3760 in place in a static state; however, a user may untwist balloon 3730 by applying sufficient force to overcome the resistive features and rotate one of elongate members 3710, 3760 in a reverse direction as that originally used to twist balloon 3730. In some embodiments, system 3700 may provide an indication to the user of how far to rotate elongate members 3710, 3760 to fully untwist balloon 3730 without going so far as to begin re-twisting the balloon in the opposing direction. For example, the detents may be arranged to provide the user with an auditory or tactile indication (e.g., a clicking sound or vibration) of how far to rotate the mechanism to untwist balloon 3730 (e.g., 10 clicks, which may correspond to the number of clicks heard by the manufacturer when originally twisting balloon 3730). Additionally, or alternatively, the mechanism and/or a proximal end of elongate members 3710, 3760 may be provided with a visual indication (e.g., a line on each elongate member 3710, 3760) of how many rotations to complete to untwist balloon 3730. In various embodiments, whatever coupler, mechanism, or other feature is keeping balloon 3730 in a twisted state may be configured to release or otherwise be overcome by forces generated in system 3200 when balloon 3730 is pressurized. As configured, balloon 3730 could be untwisted by simply inflating balloon 3730 at the treatment site.

### Additional Balloon Catheter System Embodiments

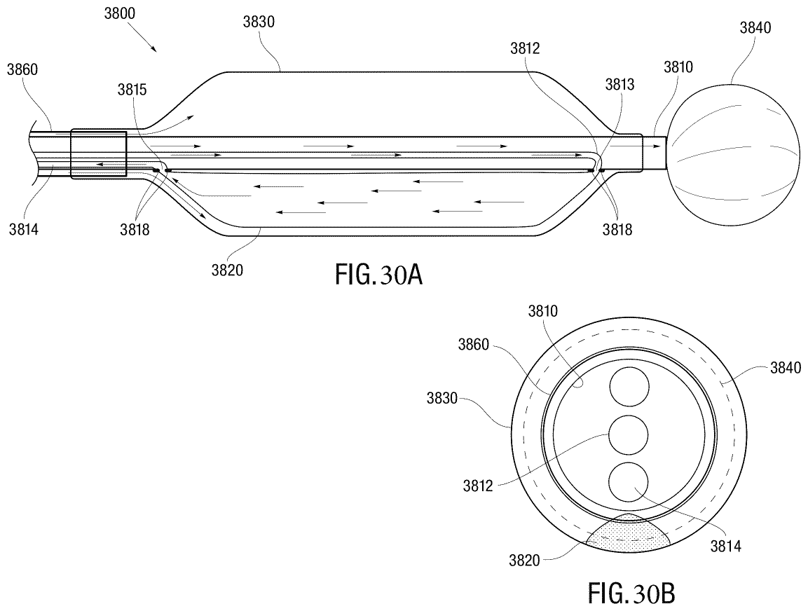

FIGS. 30A-30B, FIG. 31A-31B, and FIG. 32A-32B illustrate still further embodiments of balloon catheter systems of the present disclosure.

For example, FIG. 30A-30B illustrate a representative embodiment of system 3800 in which inner elongate member 3810 is hollow so as to house inflow and outflow conduits 3812, 3814 and to serve as a conduit, area surrounding 3812, 3814 within elongate member 3810, for inflating and deflating distal anchor balloon 3840. Similar to system 3700, in various embodiments, a distal end of balloon 3820 may be coupled to a distal end of elongate member 3810, and a proximal end of balloon 3820 may be coupled to a distal end of elongate member 3860, and an annular gap provided between elongate member 3860 and elongate member 3810 for inflating and deflating outer balloon 3830. Inflow conduit 3812 may run along an inside of hollow elongate member 3810 and exit into outer balloon 3830 through inflow port 3813 extending through the wall of hollow elongate member 3810 to feed inner balloon 3820. Outflow conduit 3814 exits outer balloon 3830 through outflow port 3815 an into the hollow interior of elongate member 3810. In the present configuration, seals 3818 may be placed at inflow port 3813 and outflow port 3815 to prevent inflation fluid (e.g., air) from escaping outer balloon 3820 into elongate member 3810, or vice versa.

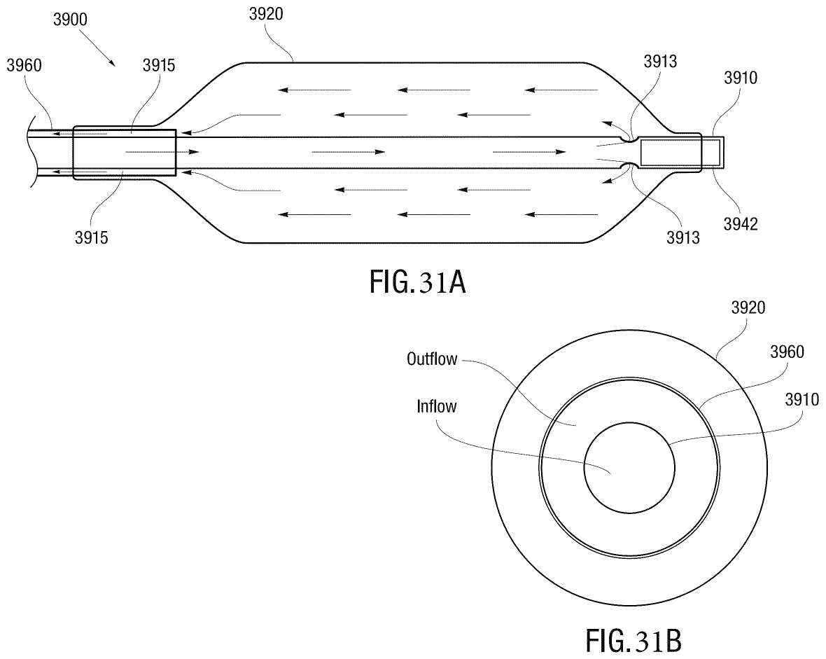

FIGS. 31A and 31B illustrate a representative embodiment of system 3900 in which elongate member 3910 can serve as inflow conduit, or flow path, and an annular gap between elongate member 3910 and elongate member 3960 serves as outflow conduit, or flow path. Such a design may be less complex than some others described herein yet maintains many of the benefits of other two-elongate-member designs previously described herein. For example, in an embodiment, elongate member 3910 may be rotated relative to elongate member 3960 to twist balloon 3920 about elongate member 3910 to streamline its profile for insertion and removal similar to system 3700 shown in FIG. 29A, FIG. 29B, and FIG. 29C. Treatment fluid may be directed to the distal end of system 3900 through a hollow interior of elongate member 3910, where it escapes into an interior of treatment balloon 3920 through inflow ports 3913. The treatment fluid may be directed back towards the proximal end of balloon 3920, where it ultimately exits balloon 3920 through the annular gap between elongate member 3960 and elongate member 3910. As shown, system 3900 may further include a plug 3942 at the distal end of elongate member 3910 to prevent treatment fluid from exiting elongate member 3910 into the body.

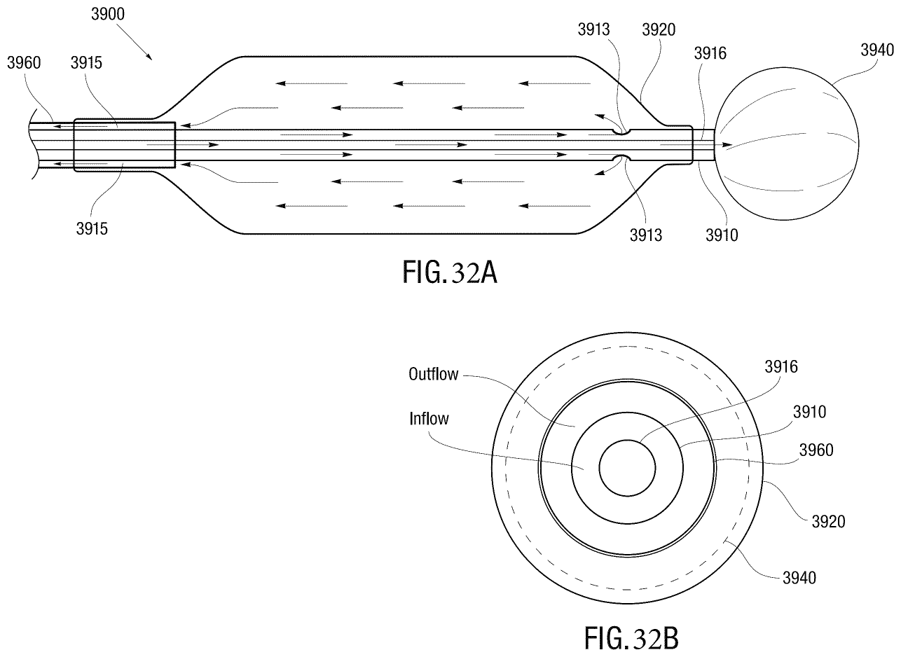

FIG. 32A-32B illustrate another representative embodiment of system 3900 including a distal anchor balloon 3940. The embodiment shown is largely the same as the embodiment shown in FIG. 31A-31B except that plug 3942 has been removed to accommodate an inflation conduit 3916 for inflating/deflating added distal anchor balloon 3940. Alternatively, a plug made of a digestible, or body compatible material, may be placed over the deflated anchor balloon 3940 and removed when anchor balloon is inflated. As shown, inflation conduit 3916 may run along the hollow interior of elongate member 3910. Treatment fluid may still be directed through elongate member 3910 through the annual gap between its walls and the walls of inflation conduit 3916.

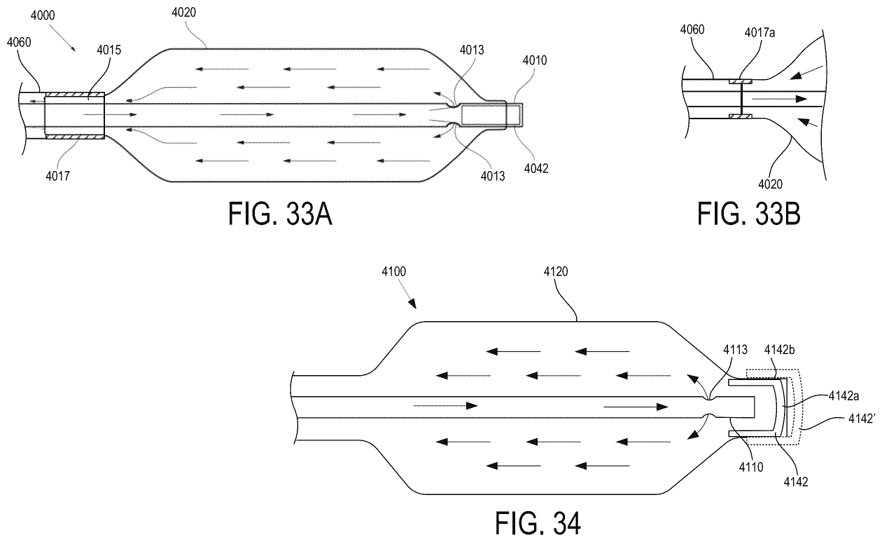

FIG. 33A illustrates a representative embodiment of another alternative system 4000. System 4000 can provide elongate member 4010 which can serve as an inflow conduit and an annular gap between elongate member 4010 and second elongate member 4060 can serve as outflow conduit 4015. In this example, treatment balloon 4020 may be coupled to elongate member 4060 via connection 4017, or collar. In some examples, treatment balloon 4020 and elongate member 4060 are coupled or bonded together via adhesive, thermal bonding, welding, or any other suitable method known in the art. Specifically, in this example, treatment balloon 4020 can be disposed inside, or within, second elongate member 4060 so that an outer surface of treatment balloon 4020 circumferentially couples or bonds to an inner surface of second elongate member 4060 at connection 4017. In some embodiments, connection 4017 can be a reinforced portion of second elongate member 4060 to provide a strengthened connection point. Without being bound to one particular theory, it is believed that mating of the treatment balloon to an inner surface of second elongate member 4060 may form a secure attachment of the two components and may permit easier inflation and/or deflation of treatment balloon 4020. For example, in one alternative embodiment, as shown in FIG. 33B, a balloon 4020 can be disposed adjacent, e.g., directly adjacent, to a second elongate member 4060 and laid end-to-end and concentric therewith. Both balloon 4020 and elongate member 4060 may be coupled to an intermediate member 4017a that is disposed inside the two components or on the outside, the intermediate member 4017a serving to couple the two components together. In at least some examples, the intermediate member 4017a is concentric with at least one or both of balloon 4020 and second elongate member 4060.

As previously described, treatment fluid may be directed to and from the distal end of system 4000 through an assembly that can be formed from a hollow interior area of elongate member 4010 and annular gap between elongate member 4010 and second elongate member 4060 can serve as outflow conduit 4015. Fluid can flow through the hollow interior area of elongate member 4010, where it can escape into an interior of treatment balloon 4020 through inflow ports 4013. The treatment fluid may be directed back towards the proximal end of balloon 4020, where it ultimately exits balloon 4020 through the outflow conduit 4015 formed from the annular gap between elongate member 4060 and elongate member 4010. It should be recognized that by spacing out inflow port 4013 and the distal end of the outflow conduit 4015 to be located near opposing ends of balloon 4020, the fluid is encouraged to travel through substantially the entire length of balloon 4020 and thereby maintain a substantially uniform temperature distribution along the length of balloon 4020. This may make it easier to control the treatment since the operator would know that all tissue adjacent to balloon 4020 is receiving the same dose of thermal energy. Conversely, were one or both of inflow port 3613 and the distal end of the outflow conduit 4015 located more towards the center of balloon 4020, fluid may stagnate and cool near opposing ends of balloon 4020, resulting in a substantially non-uniform temperature distribution along the length of balloon 4020. This may make it more difficult to control the treatment since tissue adjacent to balloon 4020 would, in turn, receive varied doses of thermal energy.

As shown, system 4000 may further include a plug 4042 at the distal end of elongate member 4010 to prevent treatment fluid from exiting elongate member 4010 into the body. FIG. 34 illustrates another representative embodiment of system 4100 that is largely the same as the embodiment shown in FIG. 33A except that a different variation of plug 4142 is disposed adjacent distal end of elongate member 4110 and translatable relative thereto. In some examples, plug 4142 may be fixedly coupled to at least one of elongate member 4110 or to balloon 4120. Alternatively, plug 4142 may reside adjacent the distal end of balloon 4120 without being coupled thereto. In the example shown, plug 4142 can be translatable relative to elongate member 4110. Plug 4142 may form an atraumatic leading tip to prevent damage to tissue and may aid in sealing and/or reinforcing portions of balloon 4120. Additionally, or alternatively, plug 4142 may aid in centering balloon 4120 with respect to elongate member 4110 to prevent damage to the balloon with movement of the elongate member. Plug 4142 may have a number of different shapes including the cup-shaped configuration shown with a base 4142a and a circumferential sidewall 4142b. Plug 4142 may be disposed so that elongate member 4110 sits at least partially within the plug (i.e., elongate member 4110 and sidewall 4142b may at least partially overlap), and contact base 4142a when pushed against the distal end of the balloon. Plug 4142 may be formed of any suitable material, including materials that are capable of bonding with polymers (e.g., PTE). In some examples, plug 4142 may be unitarily formed with the balloon, such as by tip forming the balloon and subjecting the tip of the balloon to a thermal treatment to melt and reflow the tip. In an alternative embodiment, illustrated with dashed lines, plug 4142′ is disposed on, or coupled, to an outside surface of the balloon instead of being disposed inside of it.

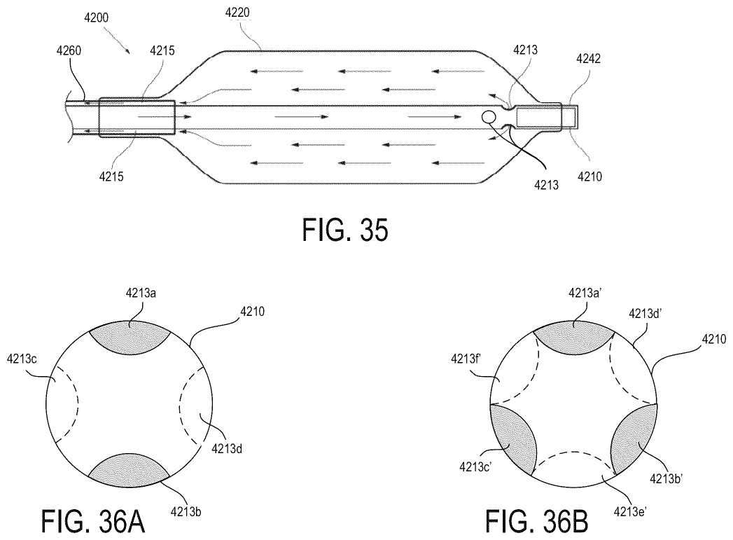

FIGS. 35, 36A, and 36B illustrate another representative embodiment of system 4200 with a further variation in the elongate member 4210. Specifically, system 4200 can include an elongate member 4210, an outer elongate member 4260, a treatment balloon 4220, an outflow conduit 4215, and a plug 4242. Except as set forth below, elongate member 4210, outer elongate member 4260, treatment balloon 4220, outflow conduit 4215, and plug 4242 are generally analogous to elongate member 3110, balloon 3120, and distal anchor balloon 3140, and system 4200 may be used in similar fashion with endoscope system 3000 as previously described in the context of systems 3100 and 3200.

Still referring to FIGS. 35 and 36A, elongate member 4210 can include a plurality of inflow ports 4213 disposed adjacent the distal end of the elongate member 4210 that are arranged in a way to maximize performance or flow through substantially the length of the balloon 4220 (e.g., to avoid cold spots in the treatment balloon). In this example, two sets of inflow ports 4213 can be formed in the elongate member, each set being disposed at a respective predetermined axial location along the elongate member 4210. In the example shown, each of the two sets of inflow ports can include two, or more, circumferentially arranged inflow ports 4213 and each of the inflow ports 4213 can be substantially circular, or semispherical, cuts in the elongate member 4210. In some examples, the inflow ports 4213 in each set of ports can be equally spaced about the circumference of the elongate member 4210. For example, a first set of two inflow ports 4213a, 4214b may be spaced 180 degrees apart and a second set of two inflow ports 4213c, 4113d can be spaced 180 degrees apart from one another, as shown in FIG. 36A. In some embodiments, the first set of two inflow ports 4213a, 4213b, and the second set of two inflow ports 4213c, 4113d, which are axially offset from the first set, can be rotated 90 degrees relative to one another. Such an arrangement, as shown in FIG. 36A, can permit for four inflow flow paths through the balloon 4220. In some embodiments, a set of three inflow ports 4213a′, 4213b′, 4213c′ may include ports that are spaced at 120 degree intervals and a second set of three inflow ports 4213d′, 4213e′, 4213f can be spaced 120 degrees apart from one another, as shown in FIG. 36B. In some embodiments, the first set of three inflow ports 4213a′, 4213b′, 4213c′, and the second set of three inflow ports 4213d′, 4213e′, 4213f, which are axially offset from the first set, can be rotated 30 degrees relative to one another. Such an arrangement, as shown in FIG. 36B, can permit for six inflow flow paths through the balloon 4220. In some embodiments, the first and second sets can include a set of four inflow ports may be 90 degrees apart. In some embodiments, the first and second sets of inflow ports can have different number of inflow ports.

In addition to the circumferential spacing between ports of a same set, different inflow port sets may be “in-phase” (e.g., circumferentially aligned) or “out-of-phase” (e.g., circumferentially misaligned or offset) with one another. As shown in FIG. 36A, a first set of inflow ports includes two inflow ports 4213a-b that are arranged 180 degrees apart from one another, and a second set of inflow ports includes two inflow ports 4213c-d that are arranged 180 degrees apart, the two sets being out of phase from one another by 90 degrees.

Turning to FIG. 36B, a first set of inflow ports includes three inflow ports 4213a′-c′ that are arranged 120 degrees apart from one another. For the sake of illustration these ports are shaded in gray. A second set of inflow ports includes three ports 4213d′-4213f, shown in dashed lines, and these ports are also arranged 120 degrees apart from one another. As shown, the two sets of ports are arranged approximately 60 degrees out of phase to create a more consistent temperature profile around the circumference of the balloon.

In some embodiments the inflow and outflow ports of any of the embodiments can have angled sidewalls to directionally influence a fluid flow passing therethrough. For example, if an inflow port is arranged at a distal end of the assembly, the angled walls can be manufactured such that the fluid is directed in a proximal direction to aid in circulating the flow of the fluid along substantially the entire length of the expandable member.

It will be appreciated that other embodiments are possible. For example, other configurations are possible including two, three, four of more sets of ports, and each of the sets may include one, two, three, four, five or six inflow ports. Additionally, a spirally-arranged, in-phase or randomly-spaced ports are also possible. In at least some examples, the two sets of inflow ports may be axially spaced apart by between 8 mm and 10 mm. Additionally, in at least some examples, the sum of the cross-sectional area of the inflow ports may be equal to or greater than the cross-sectional area of the elongate member.

### Infusion Devices

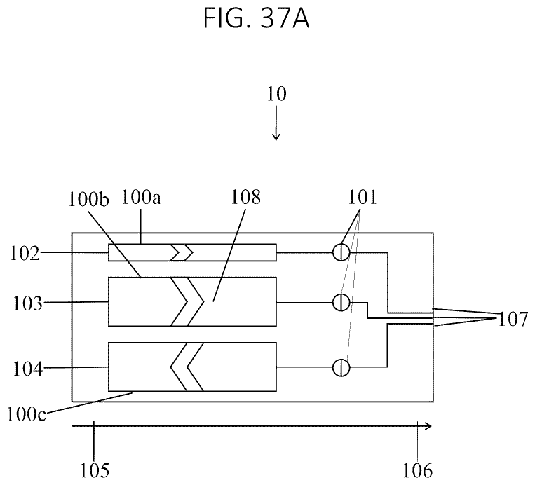

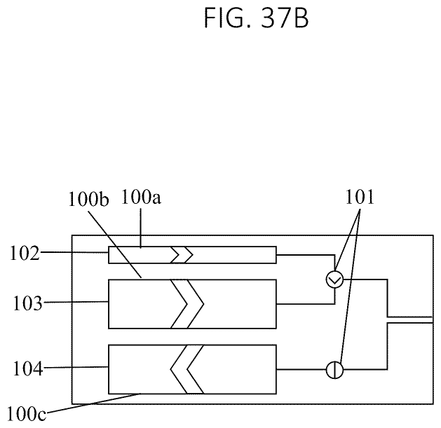

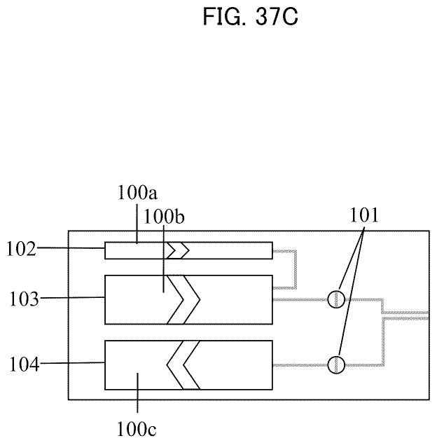

In some embodiments the system 1 can include a mechanism 10, or infusion device, having one or more fluids chambers 102-104 serving as fluid reservoirs, as shown in general in FIGS. 37A, 37B, and 37C. The embodiments disclosed in FIGS. 37A-C are shown for explanation purposes only and are not intended to limit the instant disclosure. For example, the mechanism 10, or infusion device, can include any infusion device as disclosed in U.S. Pat. No. 10,213,245, hereby incorporated by reference, in entirety.

In some embodiments, the infusion device 10 may be connected to the proximal end of an elongate member 20, as shown in FIG. 1, so that the fluid chambers 102-104 can be in fluid communication with, e.g., inflow and outflow lumens 22, 24 of the elongate member 20 and the expandable member 25. Each chamber 102-104 may communicate with the balloon 25 through its own lumen. In some embodiments, an inflation chamber 102 and inflow chamber 103 will each communicate with the balloon 25 through the same inflow lumen 22 while an outflow chamber 104 communicates through the outflow lumen 24. In some embodiments, each chamber 22-24 may have its own separate infusion device (not pictured).

In an embodiment, as shown in FIG. 37A, the fluid chambers 102, 103, 104 may include, for example, one inflation chamber 102 and two flow chambers 103, 104. The chambers 102-104 can be generally elongate structures having proximal 105 and distal 106 ends but can be of any shape. For the sake of consistency, the ends will be designated so that the distal end 106 of each chamber 102-104 communicates with the proximal end 105 of one or more of the catheter lumens 22-24. The chambers 102-104 can generally possess axial symmetry with a cross sectional profile that is most commonly circular but can also be a more complex shape. The chamber walls may include a proximal wall, a distal wall and a contiguous radial wall extending between the proximal and distal wall. The chamber walls may be rigid and may be constructed of any material compatible with the fluid to be infused, including plastic (e.g., polycarbonate, polyethylene, PEEK, ABS, nylon), glass or metal (e.g., stainless steel, aluminum, copper, brass) or some combination thereof.

In some embodiments, the inflation chamber 102 may serve as a reservoir for fluid which will be infused through the inflow lumen 22 to inflate the balloon 25 to a desired pressure and volume. The flow chambers 103, 104 may serve as reservoirs for the fluid that will continuously flow through the balloon 25 following inflation to maintain the desired therapeutic effect (e.g., constant temperature, drug concentration, etc.). For consistency, the flow chambers 103, 104 will be designated based on the direction of fluid flow relative to the balloon 25, not the chamber. Thus, the inflow chamber 103 serves as a reservoir from which fluid can be infused into the inflated balloon 25, and the outflow chamber 104 may serve as a reservoir to receive fluid that flows out of the inflated balloon 25.

Each chamber 102-104 may have one or more ports 107 through which fluid flows into (inlet port) or out of (outlet port) the chamber 102-104. Each port 107 may be associated with a valve 101 to control flow through the port 107. Each chamber 102-104 may communicate with the balloon 25 through its own lumen. In some embodiments, the infusion device 10 may have a heating mechanism 108 to heat the liquid in the inflow chamber 103. In some embodiments, the heating mechanism 108 may heat the liquid in the inflation chamber 102 so that the initial inflation can be performed with heated liquid, and in other embodiments the heating mechanism 108 may heat the liquid in the outflow chamber 104 provided the system 1 has the ability to reverse flow of the fluid and recirculate the fluid.

In some embodiments, as shown in FIG. 37B, the inflation chamber 102 and inflow chambers 103 can each communicate with the balloon 25 through the same inflow lumen 22, while the outflow chamber 104 communicates through the outflow lumen 25. In another embodiment, as seen in FIG. 37C, the inflation chamber 102 may flow into the inflow chamber 103. Each chamber 102-104 may have an infusion mechanism 100a-100c which drives fluid out of or back into the chamber 102-104 and one or more valves 101 to control the flow of fluid in and out of the chamber 102-104.

In operation, the present disclosure can be used to ablate or otherwise thermally treat a target tissue within a patient. While the instant description is described with respect to certain embodiments, one of ordinary skill in the art would appreciate that the method of operation can be used with any of the embodiments disclosed herein. Further, while this description is provided with respect to the gastro-intestinal system, one of ordinary skill in the art will appreciate that the instant system will have applicability in various other treatment locations.

An embodiment of a method of operating a system in accordance with the present disclosure, as illustrated in FIGS. 3-5, can generally include positioning, adjacent a target tissue 182 to be treated, an expandable member 3120. The system 3100 can include the expandable member 3120 and an assembly 3112 in fluid communication therewith for directing fluid into the expandable member 3120. In some embodiments, positioning can further include navigating the assembly 3112 to a desired therapeutic or target location 182 in the patient. In some embodiments, the method can further include monitoring inflation of the expandable member 3120 and monitoring the location and orientation of the expandable member 3120 relative to the target location 182. The assembly 3112 can include an input 3113 to introduce the fluid into the expandable member 3120 and an output 3115 spatially situated from the input 3113 to permit fluid flow to be removed from within the expandable member 3120. The method of operation can include infusing the fluid into the expandable member 3120, through the input 3113 of the assembly 3112, so as to enlarge the expandable member 3120 to retain the expandable member 3120 in at least one longitudinal position against the target tissue 182. Once the expandable member 3120 is expanded, the assembly 3112 can continue to direct, through the input 3113 of the assembly 3112, fluid into the expandable member 3120 and along its length to allow the fluid to transfer its energy to the tissue while simultaneously removing, through the output 3115 of the assembly 3112, fluid from the expandable member 3120, so as to maintain substantially constant fluid pressure within the expandable member 3120. Additionally, or alternatively, the expandable member 3120 can be inflated before the infusion begins.

With respect to systems 4000, 4100, and 4200, the method can include a plurality of inputs 4013, 4113, 4213a-4213d, 4213a′-4213f and any number of outputs 4015, 4215, so as to permit fluid to evenly flow through an expandable member and flow therethrough substantially along the entire length thereof. In some embodiments, as shown in FIGS. 28A and 28B, the system 3200 can include a sleeve 3260 to maintain the expandable member 3220 in a constrained configuration to permit for easier placement within the patient and the sleeve can be removed from the system 3200 as described herein.

In some embodiments, the assembly can include an elongate member 3110 and the method can additionally include advancing the elongate member 3110 through a lumen 3012 of an endoscope 3010 and rotating at least the expandable member 3120 independently of the endoscope to treat the target tissue 182 while maintaining a stationary view through the endoscope 3010. In some embodiments, the user can rotate and/or translate the elongate member 3110 relative to an endoscope 3010 for proper placement of the expandable member 3120 at the target tissue 182.

In some embodiments, for example with the expandable balloon system 3200, the method of operation can additionally include expanding an outer expandable member 3230, disposed about the expandable member 3220 to contact surrounding tissue 180 at a pressure sufficient to anchor the elongate member 3220 in place within a lumen in a patient proximate to the tissue 182 to be treated. The method can, in general, include transferring thermal energy from the fluid directed into and out of an interior of the expandable member 3220 to treat the target tissue 184 through the expandable member 3220 and/or the outer expandable member 3230.

In some embodiments, the system, e.g., system 3200 as shown in FIG. 16, can further include a distal balloon 3240, and the method of operation can additionally include expanding the distal balloon 3240 to contact surrounding tissue at a pressure sufficient to anchor the expandable member 3230 in place within the body, or expanding the distal balloon 3240 to dimensions greater than a diameter of a passageway within the body such that, when positioned beyond the passageway and expanded, such that the distal balloon 3240 is prevented from being pulled back through the passageway.

In some embodiments, as shown for example in FIGS. 20A, 20B, 21A, 21B, the method can include rotating the expandable member 3320 independent of the outer expandable member 3330 to treat additional tissue about a circumference of the lumen. Further, in some embodiments, the method can include translating the expandable member 3320 relative to the outer expandable member 3330 to treat additional tissue 184 along a length of a tract. This relative motion of the expandable member 3320 within the outer expandable member 3330 can allow for the outer expandable member 3330 to anchor the system within a lumen of the patient while allowing for targeted thermal treatment of the tissue 184 without the need to deflate the outer expandable member 3330 to treat a different area of tissue 184.

Generally, the instant method can be used for treating tissue in an upper gastrointestinal tract; tissue in a lower gastrointestinal tract; parasympathetic nerves in a renal artery; parasympathetic nerves in a nasal cavity; treating vessels in a circulatory system; treating fistulae tracts; breast tissue, and/or other treatment areas necessitating thermal treatment.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A system for treatment of tissue, the system comprising,
an expandable member designed to be positioned at a site of interest against tissue to be treated and to accommodate fluid set at a temperature range to affect the tissue at the site of interest;
an assembly for directing the fluid set at the temperature range into the expandable member, the assembly having an input to introduce the fluid into the expandable member to circulate substantially along an entire length of the expandable member and an output spatially situated from the input to permit fluid flow to be removed from within the expandable member; and
a mechanism designed to direct fluid along the assembly and through the input into the expandable member while simultaneously removing fluid from the expandable member through the output, so as to maintain substantially constant fluid pressure within the expandable member.

2. The system of claim 1,
wherein the assembly includes an input fluid pathway and an output fluid pathway, and
wherein the input fluid pathway extends from the mechanism to the input and the output fluid pathway extends from the mechanism to the output.

3. The system of claim 2, wherein the input fluid pathway is disposed concentrically with the output fluid pathway.

4. The system of claim 3, wherein the output fluid pathway is arranged between an inner surface of an elongated member and an outer surface of the input fluid pathway.

5. The system of claim 4, wherein
the input fluid pathway extends into the expandable member to deliver fluid into the expandable member, and
a proximal end of the expandable member is partially disposed within the output fluid pathway.

6. The system of claim 5, wherein the proximal end of the expandable member is adhered to an inner surface of the elongated member.

7. The system of claim 5, wherein a portion of the input fluid pathway that is disposed within the expandable member defines a plurality of inflow ports adjacent a distal end of the input fluid pathway.

8. The system of claim 7, wherein the plurality of inflow ports are arranged in a plurality of sets, each of the plurality of sets being axially spaced from others of the plurality of sets.

9. The system of claim 8, wherein each of the plurality of sets is circumferentially misaligned with adjacent ones of the plurality of sets.

10.-17. (canceled)

18. A method of treating tissue, comprising:
positioning, adjacent a target tissue to be treated, an expandable member having an assembly in fluid communication therewith for directing fluid into the expandable member, the assembly having an input to introduce the fluid into the expandable member and an output spatially situated from the input to permit fluid flow to be removed from within the expandable member;
infusing fluid into the expandable member, through the input of the assembly, so as to enlarge the expandable member to retain the expandable member in at least one longitudinal position against the target tissue; and
continuing to direct, through the input of the assembly, fluid into the expandable member and along its length to allow the fluid to transfer its energy to the tissue while simultaneously removing, through the output of the assembly, fluid from the expandable member, so as to maintain substantially constant fluid pressure within the expandable member.

19. The method of claim 18,
wherein the assembly includes an elongate member and the method further comprises,
advancing the elongate member through a lumen of an endoscope, and
rotating at least the expandable member independent of the endoscope to treat the target tissue while maintaining a stationary view through the endoscope.

20. The method of claim 19, the method further comprising expanding an outer expandable member, disposed about the expandable member to contact surrounding tissue at a pressure sufficient to anchor the elongate member in place within a lumen in a patient proximate to the tissue.

21. The method of claim 20, further including rotating the expandable member independent of the outer expandable member to treat additional tissue about a circumference of the lumen.

22. The method of claim 20, further including translating the expandable member relative to the outer expandable member to treat additional tissue along a length of a tract.

23. The method of claim 20,
wherein thermal energy from the fluid directed into and out of an interior of the expandable member treats the target tissue through the expandable member and the outer expandable member.

24. The method of claim 20,
wherein the expandable member further includes a distal balloon, and
wherein the method further includes either:
expanding the distal balloon to contact surrounding tissue at a pressure sufficient to anchor the expandable member in place within the body, or
expanding the distal balloon to dimensions greater than a diameter of a passageway within a body such that, when positioned beyond the passageway and expanded, such that the distal balloon is prevented from being pulled back through the passageway.

25.-31. (canceled)

32. A system for treatment of tissue, comprising:
an expandable member sized to be positioned against tissue to be treated and to accommodate fluid set at a range of temperatures to affect the tissue;
an assembly extending into the expandable member for directing the fluid thereinto, the assembly comprising,
a first fluid path terminating at an input so as to permit fluid introduction into the expandable member to circulate along its length;
a second fluid path terminating in an output, and is spatially situated from the input to permit circulated fluid flow to be removed from within the expandable member; and
a mechanism, in fluid communication with the assembly, designed to push fluid through the input into the expandable member while simultaneously removing fluid from the expandable member through the output, so as to maintain substantially constant fluid pressure within the expandable member.

33. The system of claim 32, wherein the expandable member is disposed within an inner surface of the second fluid path.

34. The system of claim 32, wherein the second fluid path is disposed around the first fluid path and is concentric with the first fluid path.

35. The system of claim 32, wherein a portion of an outer surface of the expandable member is adhered to an inner surface of the second fluid path.

36.-45. (canceled)
